(19) United States
(12) Patent Application Publication
(10) Pub. No.: US 2019/0211074 A1
(43) Pub. Date: Jul. 11, 2019

(54) Methods for protein modification in pharmaceutical applications

(71) Applicant: Tianxin Wang, Walnut Creek, CA (US)

(72) Inventor: Tianxin Wang, Walnut Creek, CA (US)

(73) Assignee: Biotether, San Rafael, CA (US)

(21) Appl. No.: 16/364,113

(22) Filed: Mar. 25, 2019

Related U.S. Application Data
(63) Continuation of application No. 15/169,640, filed on May 31, 2016, now abandoned.
(60) Provisional application No. 62/174,528, filed on Jun. 12, 2015.

(51) Int. Cl. C07K 14/61; C07K 16/32; C07K 16/10; C07K 16/00; C12N 9/00

(52) U.S. Cl. CPC C07K 14/61; C07K 16/32; C07K 16/1063; C07K 16/00; C12N 9/00; C07K 2317/24; C07K 2319/50; C07K 2319/30; C07K 2319/33; C07K 2317/622; C07K 2319/61; C07K 2319/70; C07K 2317/76

(57) ABSTRACT

This disclosure provides methods to modify protein for pharmaceutical applications and reagents to treat disease such as pathogen infection and cancer. The method involves increasing the molecular weight of the protein by connecting multiple protein units with site specific conjugation to extend the in vivo half life. This disclosure also provides methods to construct affinity ligand in protein or aptamer form, which becomes active when they reach the treatment target, therefore provide higher specificity for treatment.

### CROSS-REFERENCE TO RELATED APPLICATIONS

This application is a continuation application of U.S. patent application Ser. No 15/169,640 filed on May 31, 2016, which claims priority to U.S. Provisional Patent Application No. 62/174,528 filed on Jun. 12, 2015. The entire disclosure of the prior application is considered to be part of the disclosure of the instant application and is hereby incorporated by reference.

### BACKGROUND OF THE INVENTION

### Field of the Invention

The current invention relates to methods to modify protein for pharmaceutical applications and reagents to treat disease such as pathogen infection and cancer. The current invention also relates to methods to extend the in vivo half life and potency of protein and aptamer based reagents.

### Background Information

Protein drugs have changed the face of modern medicine, finding application in a variety of different diseases such as cancer, anemia, and neutropenia. As with any drugs, however, the need and desire for drugs having improved specificity and selectivity for their targets is of great interest, especially in developing second generation of protein drugs having known targets to which they bind. It is also desirable to have a long in vivo half life for the protein drug to reduce their injection frequency to provide a better treatment for patient. Extending the half-life a therapeutic agent, whether being a therapeutic protein, peptide or small molecule, often requires specialized formulations or modifications to the therapeutic agent itself. Conventional modification methods such as pegylation, adding to the therapeutic agent an antibody fragment or an albumin molecule, suffer from a number of profound drawbacks. For example, PEGylated proteins have been observed to cause renal tubular vacuolation in animal models. Renally cleared PEGylated proteins or their metabolites may accumulate in the kidney, causing formation of PEG hydrates that interfere with normal glomerular filtration. Thus, there remains a considerable need for alternative compositions and methods useful for the production of highly pure form of therapeutic agents with extended half-life properties at a reasonable cost.

### BRIEF DESCRIPTION OF THE DRAWINGS

FIG. 1 shows multivalent homo Fab format with suitable length flexible linker for higher affinity.

FIG. 2 shows hetero Fab format targeting two antigens of the different protein on the cell/microorganism for higher affinity.

FIG. 3 shows Hetero Fab format targeting two epitope sites of the same target protein for higher affinity.

FIG. 4 shows construction of bi-specific antibody and ADC using selective reduction.

FIG. 5 shows bi specific antibody by linking two or more full size antibodies.

FIG. 6 shows an example of the preparation of bi specific antibody by linking two full size antibodies.

FIG. 7 shows uses an example of using immobilized affinity group targeting the carbohydrate on the antibody to selectively protect one FC conjugation site on the antibody to achieve mono conjugation

FIG. 8 shows mono labeling of drug and linker on the antibody

FIG. 9 shows the structure and activating mechanism of self assembly probody

FIG. 10 shows examples of self assembly probody with Fc modifier

FIG. 11 shows the activation mechanism of self assembly probody with Fc modifier

FIG. 12 shows an example of self assembly probody with Fc modifier

FIG. 13 shows example of self assembly probody with heterogenic MM

FIG. 14 shows the structure and activating mechanism of protamer

FIG. 15 shows the structure and activating mechanism of self assembly protamer

FIG. 16 shows examples protamer with half life modifier or drug conjugation

FIG. 17 shows an example of Binding Based Prozyme, which is an enzyme activated upon binding of aptamer

FIG. 18 shows an example of Binding Based Prozyme, which is an enzyme activated upon binding of antibody

FIG. 19 shows the scheme of ABP (antibody binding partner)-linker-EIP (enzyme inhibition partner) based Prozyme

FIG. 20 shows the examples of format of ABP (antibody binding partner)-linker-EIP (enzyme inhibition partner) based prozyme

FIG. 21 shows the scheme of Cleavage Based Prozyme, which is an enzyme activated with second enzyme

FIG. 22 shows an examples of a block polymer made of two PEG blocks connected with a biodegradable polylactic acid.

FIG. 23 shows different formats of biodegradable PEG and the biodegradable HGH dimer.

FIG. 24 shows an example of HGH trimer that can extend HGH in vivo half life.

FIG. 25 shows an example of the HGH trimer and its preparation

FIG. 26 shows an example of HGH trimer using 3 arm linker

FIG. 27 shows another example of HGH trimer using 3 arm linker

FIG. 28 shows the scheme of crosslink HGH with affinity group to extend its in vivo half life

FIG. 29 shows the scheme of crosslink HGH with antibody to extend its in vivo half life

FIG. 30 shows HGH trimer for half-life extension using a small PEG or peptide as linker and the synthesis.

FIG. 31 shows another example of HGH trimer for half-life extension using a small PEG as linker and the synthesis.

FIG. 32 shows examples of HGH oligomer with biodegradable linker.

FIG. 33 shows an example of HGH oligomer with peptide linker prepared with recombinant technology.

FIG. 34 shows examples of HGH oligomer with terminal modifier.

### DESCRIPTION OF THE INVENTIONS AND THE PREFERRED EMBODIMENT

The current invention discloses a method and formulation dosage form to improve the in vivo half life and potency of biological active protein by combining protein with protein-antibody immuno complex and administering it to the patient, in which the amount of protein is greater than the binding capacity of antibody to provide free unbound protein in the formulation. In the current inventions the “/” mark means either “and” or “or”.

The method comprises the following steps:

1) Administering protein-antibody immuno complex to the patient at the effective amount for desired biological activity of the protein. This can be achieved by prepare the protein-antibody immuno complex first and then administer it to the patient. Optionally the mixture of free additional protein and the protein-antibody immuno complex can be used instead of protein-antibody immuno complex only. This can also be done by administering protein and the antibody separately to the patient to allow the formation of immuno complex in vivo. In some embodiments the amount of the protein is equal or greater than the binding capacity of the antibody. For example, if IgG is used, the amount of the protein is no less than two times of the antibody amount (molar ratio) because each IgG binds with two proteins. In these embodiments all the binding sites of antibody in the protein-antibody immuno complex are bound with protein. Examples of suitable administering routes include intravenous, intraperitoneal, intramuscular and subcutaneous routes and their combinations.

2) After a certain period of time when the in vivo concentration of the protein decreases to undesired level, enough amount of protein is re-administered to the patient to maintain the desired in vivo protein concentration (free and bound form). Sometimes additional protein-antibody immuno complex can also be administered together with the protein to maintain the desired in vivo antibody concentration, which results in desired protein-antibody immuno complex concentration to ensure the sustained desired in vivo protein concentration.

3) Step 2 can be repeated several times based on the required in vivo protein concentration and treatment length. For example, step 2 is repeated every 7 days or every 10 days or every two weeks or every 20 days or every month for 3 months or 6 moths or 1 year or a few years.

The current invention discloses pharmaceutical formulation forms suitable for above method. The pharmaceutical formulation form contains two or more dose, the first dose contains effective amount of protein-antibody immuno complex or the mixture of free (unbound) protein and the protein-antibody immuno complex. The second and later doses contain suitable amount of protein drug only or the mixture of free protein and the protein-antibody immuno complex.

It is know that when antigen binds with antibody, the half life of the immuno complex can be longer than that of the antigen alone, therefore provide longer in vivo half life, which is useful for increase protein drug potency and reduce elimination. The antibody can also protect the protein from enzyme degradation which also increase its half life and potency. However, the dissociated protein has much faster clearance rate than the antibody therefore after the injection of immuno complex, the ratio of protein vs antibody become smaller overtime and the concentration of the protein decrease in a much greater extent than the decrease of antibody. The unbound antibody will inhibit the protein activity, which further reduce the protein activity in vivo. Repetitive injection of immuno complex will further increase the unbound antibody concentration which will become an antibody trap therefore cannot provide satisfactory in vivo protein activity for treatment. The current invention solves this problem by injecting free protein only or mixture of free protein with the protein-antibody immuno complex, to maintain the desired protein concentration without causing the buildup of antibody in vivo.

For example, a protein P (30 KD) is used for treating certain disease. Antibody IgG Abp (150 KD) is its neutralizing antibody. Using the method of the current invention, 6 mg of P is mixed with 15 mg Abp to prepare the immuno complex P-Abp in which each Abp binds with 2 P. At the beginning of the treatment, 21 mg of P-Abp is injected (i.V.) to the patient. The in vivo half life of P is 10 d and 20 d for Abp and 70% Abp left on day 10 (based on the concentration of both free form and bound form in immuno complex after administering P-Abp). The in vivo half life of P not in immuno complex is 0.5 d. Therefore, a second dose containing 3-6 mg of P is given on day 10 and a third dose containing 10.5 mg of P-Abp and 3 mg of P is given on day 20 to maintain a steady effective in vivo protein P concentration. This can be repeated until the treatment is finished (e.g. another second dose on day 30 and another third dose on day 40). The formulation form will contain 1 first dose (21 mg of P-Abp) and multiple second doses (3-6 mg of P) and multiple third doses (mixture of 10.5 mg of P-Abp and 3 mg of P).

Alternatively, 21 mg of P-Abp is injected (i.V.) to the patient at the beginning of the treatment, and a mixture of 3 mg of P with 6.3 mg Abp-P (30% of 21 mg because 30% of Abp is cleared on day 10) is injected every 10 days. The formulation form will contain 1 first dose (21 mg of P-Abp) and multiple second doses (mixture of 3 mg of P with 6.3 mg P-Abp). If high dose of free P does not cause adverse effect, 3× second doses can be injected on day 1 instead of 21 mg P-Abp, therefore the drug formulation only need to contain multiple of mixture of 3 mg of P with 6.3 mg P-Abp.

Besides what listed above, other scheme of the administering dose/interval and formulation composition can be used to achieve the desired in vivo P concentration. The pharmacokinetics (e.g. in vivo half life) can be measured for each individual to prepare the personalized medical treatment. Averaged pharmacokinetics data from a large population can also be used instead to design the composition of the formulation and administering schedule. Other route (e.g.

subcutaneous or intramuscular injection) can also be used.

There are many protein drugs can be used according the above method described in the current invention. For example, HGH and antibody against HGH (either neutralizing IgG or non neutralizing IgG, the best antibody can be obtained by screening), IL-7 and M25 antibody, human IL-10 (hIL-10) and humanized antihuman IL-10 (hαhIL-10) can be used for the current application.

In some embodiments, the antibody-antigen protein drug complex used has a molar ratio of antibody : antigen>0.5, which means some of the antibody binding sites do not bind with antigen protein drug, to achieve a more steady blood drug concentration change. For example, antibody bound with antigen at 1:1 ratio (half of the binding sites are empty in each antibody) is used as antibody-antigen drug immuno complex. In one example, the first dose of is the antibody bound with antigen drug at 1:1 ratio, the second and later dose contains two parts: free antigen drug and antibody bound with antigen drug at 1:1 ratio. The two parts can be injected at the same time or sequentially.

Example: Development Plan for HGH Affinity Dosing

1) Antibody Screening:

Mix several antibody (monoclonal from mouse, many commercially available) against HGH with HGH and inject to the mouse

Measure the serum HGH level and select the antibody that extend the HGH half-life the most

2) Dosing Screening :

Adjust the ratio between Ab: HGH of the first dose to select the one providing the best PK profile after the first dose

Adjust the ratio between Ab: HGH of the later doses to select the one providing the best PK profile (or weight gain) during the later dose, the adjustment can be designed based on the pK model developed during screening.

3) Humanizing: Antibody humanization and Dosing Adjustment for Human

The current invention also discloses novel strategy for site specific conjugation of proteins including antibodies. Site specific antibody drug conjugation is a promising drug discovery strategy for cancer treatment; several companies (e. g. ambrx, innate-pharma and sutrobio) are working on developing new method for site specific conjugation of proteins, In one aspect, the new method in the current invention uses elevated temperature for site specific conjugation using MTgase (microbial transglutaminase, also called bacterial transglutaminase, BTG) to couple the drug/linker having amine group to the Gln of the protein. Preferred temperature is >40 degree, more preferably >45 degree but less than 75 degree. In some embodiments, the temperature is 50˜65° C. The elevated temperature can expose the previous hidden (e.g. the Gln in antibody difficult to be accessed by MTgase) functional groups for site specific conjugation.

In one example conjugation of IgG1 with Monodansylcadaverine (MDC) is catalyzed by MTgase. MDC has a primary amine and its fluorescence can be easily monitored. MDC is used here to conjugate to mAB. To purified IgG1 (1-10 mg/ml) in Tris-buffer (pH 6.5-8.5), add MDC (Sigma-Aldrich) in DMSO to final concentrations of 1-5 mM (final DMSO 2-10%). Add purified MTgase to a final concentration of 0.05-1.0 mg/ml. Incubate the reaction mixtures at 50° C. for 5 hours. Reaction is monitored by HPLC. Antigen peptide for the IgG (e.g. 5 fold excess) can be added to the reaction mix to stabilize the Fab of the antibody.

In another aspect, the new method in the current invention uses MTgase to couple the drug/linker having Gln group to the amine group of the protein (e.g. lysine or N terminal amine). The coupling can be done in either high temperature (e.g. 45˜55° C). or low temperature (e.g. 25-37° C.). Point mutation can be used on the protein (e.g. antibody) to introduce lysine as coupling site.

In one example, pegylation of IgG1 with 1 kDa PEG-CO-Gln-COOH or PEG-CO-Gln-Gly-NH2 is performed by MTgase catalysis. This experiment is carried out essentially the same condition as described in the example above. The MDC is replaced with MW=1 k PEG-CO-Gln-COOH (the product of HO-PEG-COOH coupling with Gln, which for an amide bond between PEG-COOH and the amine of Gln) or PEG-CO-Gln-Gly-NH2 in pH 7.0 to a final concentration of 1 to 2 mM, PEGylated IgG1 is obtained. The Gln of on the PEG couples to the amine group on the IgG1 by MTgase catalysis.

The current invention also discloses novel toxin which can be used for antibody-drug conjugate (ADC) and cancer treatment. Currently MMAE (monomethyl auristatin E) or MMAF is used for ADC as toxin to conjugate with antibody. The novel toxins in the current invention are N-substituted MMAE/MMAF. Their structures are shown below (the attachment group is where the toxin to be conjugated with):

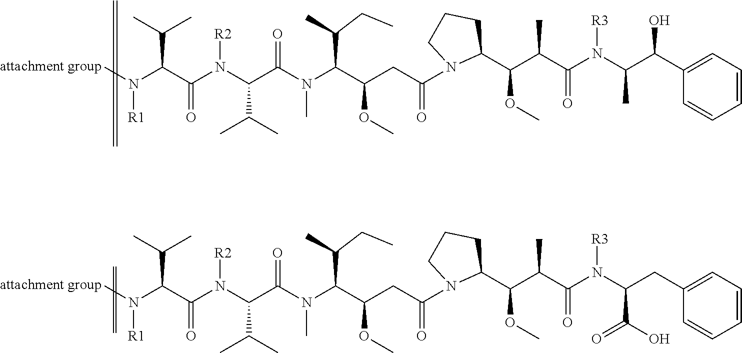

Where in R1, R2 and R3 is independently selected from the group consisting of H, C1-C8 alkyl, haloC1-C8 alkyl, C3-C8 carbocycle, aryl, X-aryl, OR21, SR21, N(R21)2, —NHCOR21 and —NHSOR2R21, X—(C3-C8 carbocycle), C3-C8 heterocycle and X—(C3-C8 heterocycle), each X is independently C1-C10alkylene.

In some examples, R1 is independently H or CH3 or CH2F or CHF2 or CF3, R2 independently is H or CH3 or CH2F or CF3 and R3 is independently H or CH3 or CH2F or CF3.

The structures also include:

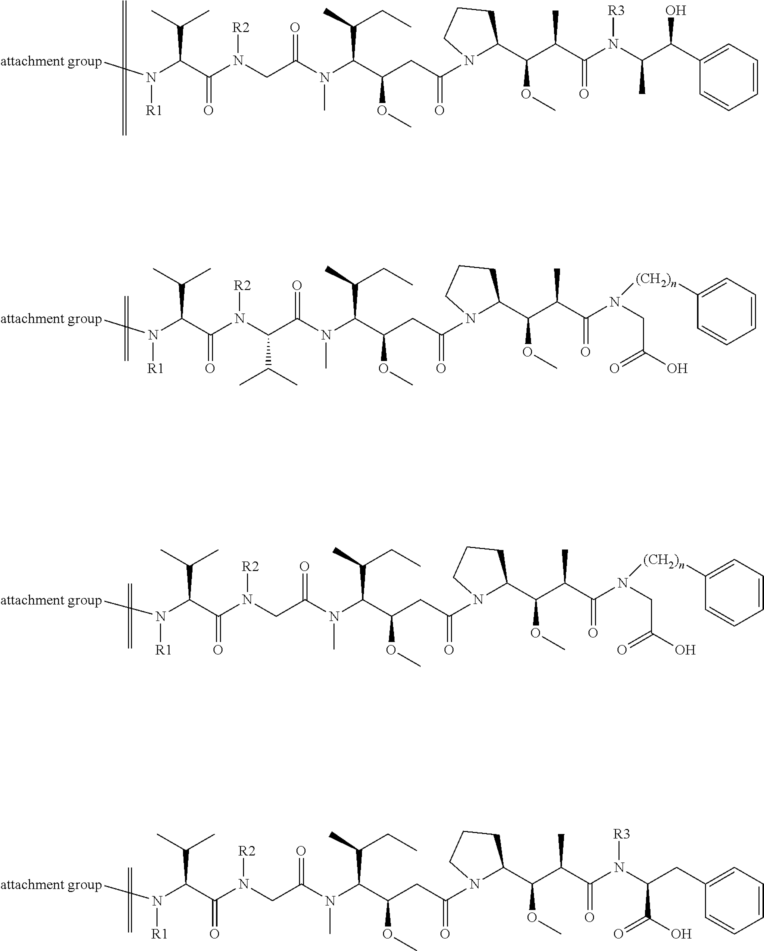

Where in R1, R2 and R3 is independently selected from the group consisting of H, C1-C8 alkyl, haloC1-C8 alkyl, C3-C8 carbocycle, aryl, X-aryl, OR21, SR21, N(R21)2, —NHCOR21 and —NHSOR2R21, X—(C3-C8 carbocycle), C3-C8 heterocycle and X—(C3-C8 heterocycle), each X is independently C1 -C10alkylene, n is an integer between 1˜5.

In some examples, R1 is independently H or CH3 or CH2F or CHF2 or CF3, R2 independently is H or CH3 or CH2F or CF3 or isopropyl and R3 is independently H or CH3 or CH2F or CF3.

The attachment group is where the toxin conjugates to linker or proteins. It is the same as those used in the current MMAE/MMAF ADC.

The current invention also discloses novel strategy for antibody purification and conjugation. Current antibody purification method uses protein A column, which is expensive and has 250 potential risk of leaking protein A. The new strategy uses affinity column based on epitope peptide or mimotope for antibody purification by coupling epitope peptide or minotope to the solid phase support as column filler, e.g. sephadex beads. The advantages are low cost, more stable chemistry for immobilization, selectively isolating antibody with high binding affinity and removing non binding antibody/ADC, therefore increase the potency and therapeutic index of antibody or ADC. In one example: peptide NIYNCEPANPSEKNSPSTQYCYSI (SEQ ID NO: 1) is used to couple to solid phase support to make an affinity column, which can be used for Rituximab purification. The benefit of using peptide based affinity column (activated beads are commercially available) is greater than the effort of developing the peptide for each antibody. Many peptide sequence are available from literature or epitope scan for both linear and conformational discourteous epitope (e.g. from pepscan). This strategy also works for other protein drugs by using synthetic ligand (e.g. affinity peptide) for the binding site of that protein to prepare affinity column.

Furthermore, it can be used to selectively protect the reactive amino acid in the binding site of the antibody, by adding epitope peptide or mimotope (free form or immobilized) or masking peptide (e.g. those used in probody) to form the peptide-antibody complex during antibody-drug conjugation. Similarly it can be used to protect the active binding site of other type of protein by using the affinity ligand that can mask the active binding site of that protein. This method is suitable for both chemical and enzymatic conjugation, therefore provide more drug load for ADC, more conjugation reaction can be allowed (e.g. >2 types of toxin). Similar strategy is used in enzyme conjugation to keep the enzyme activity by adding enzyme substrate. Synthetic peptide is very easy to make (low cost and more stable) using synthetic peptide chemistry than making proteins. Peptide can be made in large amount easily using solid phase peptide synthesis. In one example: peptide NIYNCEPANPSEKNSPSTQYCYSI (SEQ ID NO: 1) is used to protect Rituximab during conjugating drugs to the antibody. Peptide NIYNCEPANPSEKNSPSTQYCYSI (SEQ ID NO: 1) can bind with Rituximab at its antigen binding site. By adding NIYNCEPANPSEKNSPSTQYCYSI (SEQ ID NO: 1) (preferably at >2:1 ratio) to Rituximab before chemical conjugation on Rituximab, the antigen binding site of Rituximab is protected.

The current invention also discloses novel Bi specific antibody and its application. They can be used to treat cancer, pathogens, immune disorders and targeting delivery of vector (retrovirus based gene therapy).

Bi specific antibody can be in traditional monomer format: multivalent homo Fab format with a suitable length flexible linker for higher affinity (not bi specific), hetero Fab format targeting two epitope sites of the different protein on the cell/microorganism to achieve higher affinity and hetero Fab format targeting two epitope sites of the target protein to achieve higher affinity.

Bi specific antibody can also be in dimer format or trimer or higher degree oligomer format: multivalent homo Fab format with suitable length flexible linker for higher affinity (not bi specific), hetero Fab format targeting two epitope sites of the target protein for higher affinity and hetero Fab format targeting two epitope sites of the different protein on the cell/microorganism for higher affinity. Construction of this type of Bi specific antibody can be achieved using boric affinity column or lectin affinity column for mono conjugation (boric affinity column or lectin affinity column can also be used for antibody purification).

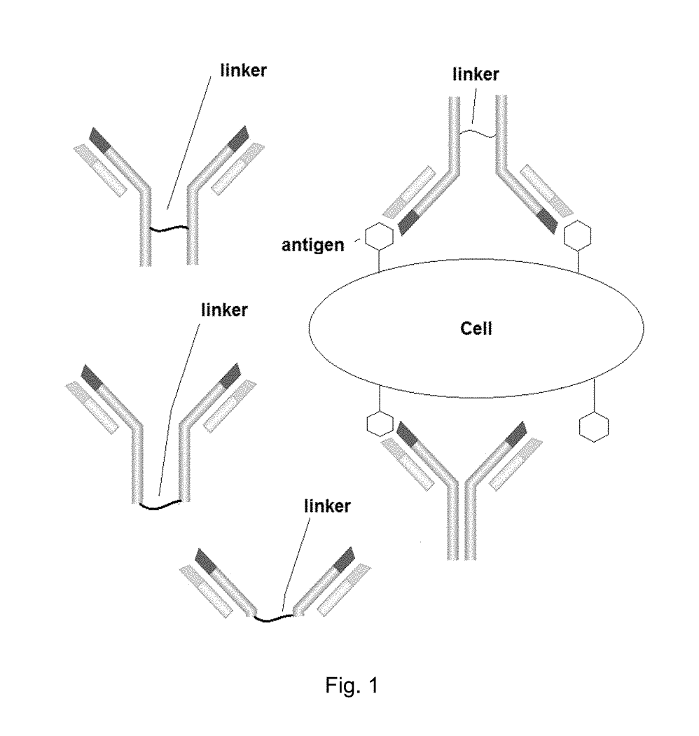

Bi Specific Antibody (BsAb) can be used for against cytoplasm target. In some embodiments, Bi specific antibody is in traditional antibody monomer format: multivalent homo Fab format with suitable length flexible linker for higher affinity. Native antibody's hinge region is not long and flexible enough therefore may not reach two antigens on the target cell. Using a flexible and suitable length of linker to connect the antibody parts will greatly increase the binding affinity (FIG. 1). The linker can be a flexible peptide linker such as poly glycine/serine or synthetic polymer such as PEG. In the current inventions the “/” mark means either “and” or “or”.

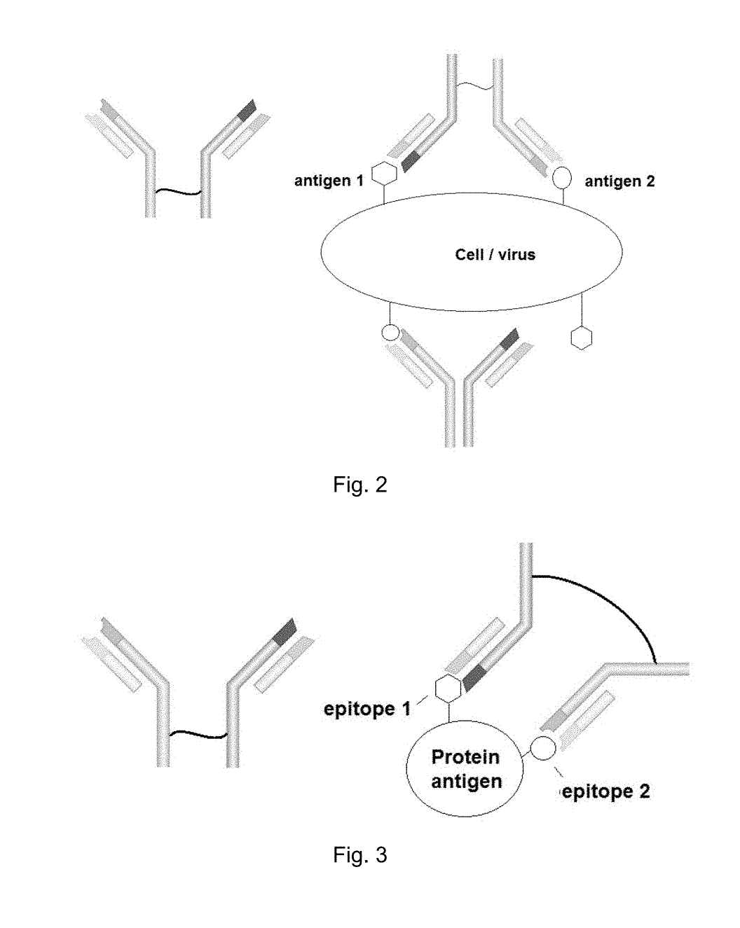

It can also be hetero Fab format targeting two antigens of the different protein on the cell/microorganism for higher affinity. Similarly, the above approach can also be applied to bispecific antibody binding to two different antigens on the cell/pathogen. The bispecific antibodies with flexible proper length linkers can be made easily to get the optimal binding of two antigens simultaneously while traditional method is time consuming (FIG. 2).

Another format is to use bi specific antibody to target the two different epitopes on the same antigen, which will also significantly increase the binding affinity (FIG. 3).

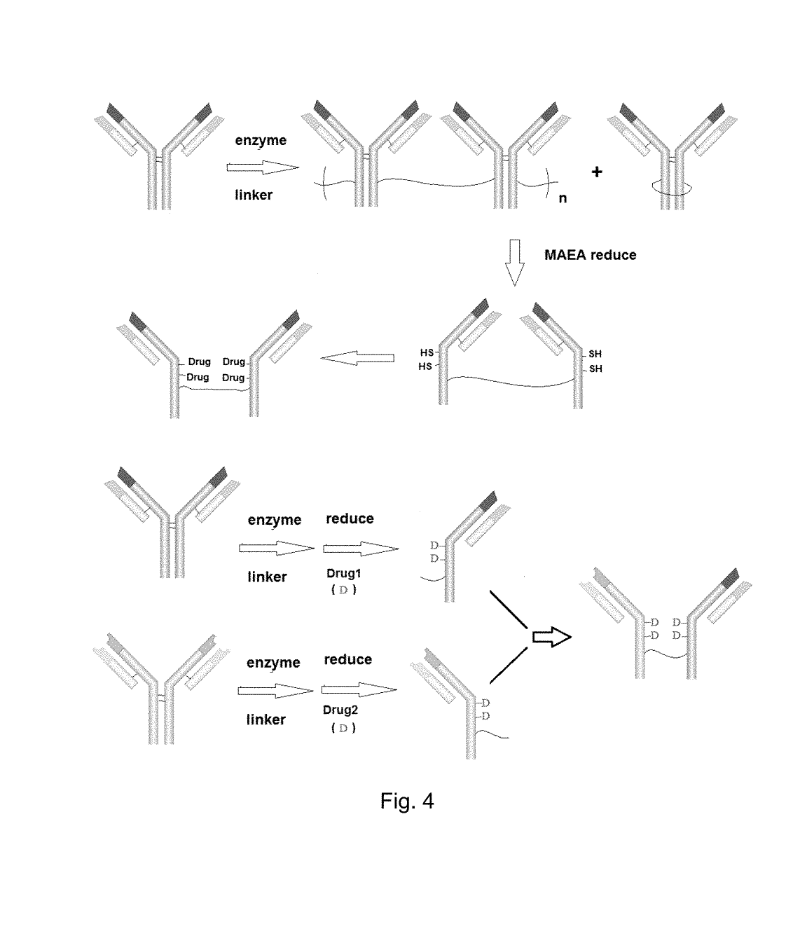

Construction of these types of Bi specific antibody: Using the selective reduction of the disulfide bond at the hinge region with 2-Mercaptoethylamine , several formats (FIG. 4) can be used to make this type of bispecific antibodies, with high yield and no concern for dimer formation to ease the industrial scale separation process. Two formats are shown below: to use some —SH reactive reagent (or mutation to remove —SH) to block the free —SH group to prevent the regeneration of —SS-bond, which will generate the traditional format bispecific antibody.

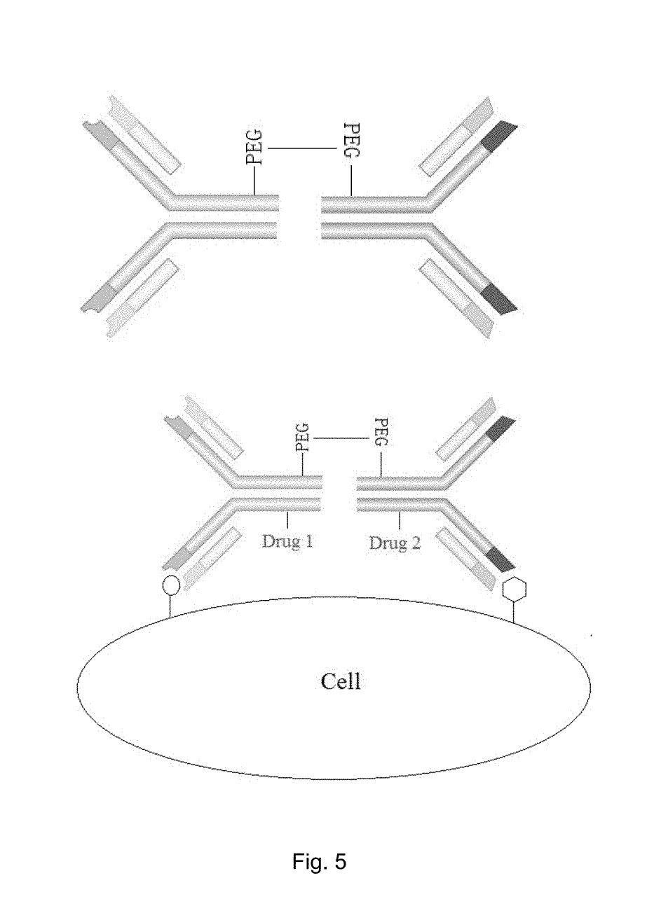

Similarly, bi specific antibody by linking two or more full size antibodies can also be used in above applications (FIG. 5) and formats and synthesized readily (FIG. 6), which may offer higher stability and higher binding affinity as shown by IgA and IgM.

Construction of this type of Bi specific antibody can be achieved using borate affinity column or lectin affinity column for mono conjugation. This strategy is also useful for antibody purification. This design uses immobilized antibody to archive high yield mono labeling of the antibody, to eliminate the potential bi-labeled antibody (generating polymerized antibody).

Immobilized protein was used to make mono PEGlated protein previously. Ion exchange resin was used to immobilize the protein. However ion exchange resin may not work for antibody to block half of FC and the binding affinity is low, which may cause exchange between two sides.

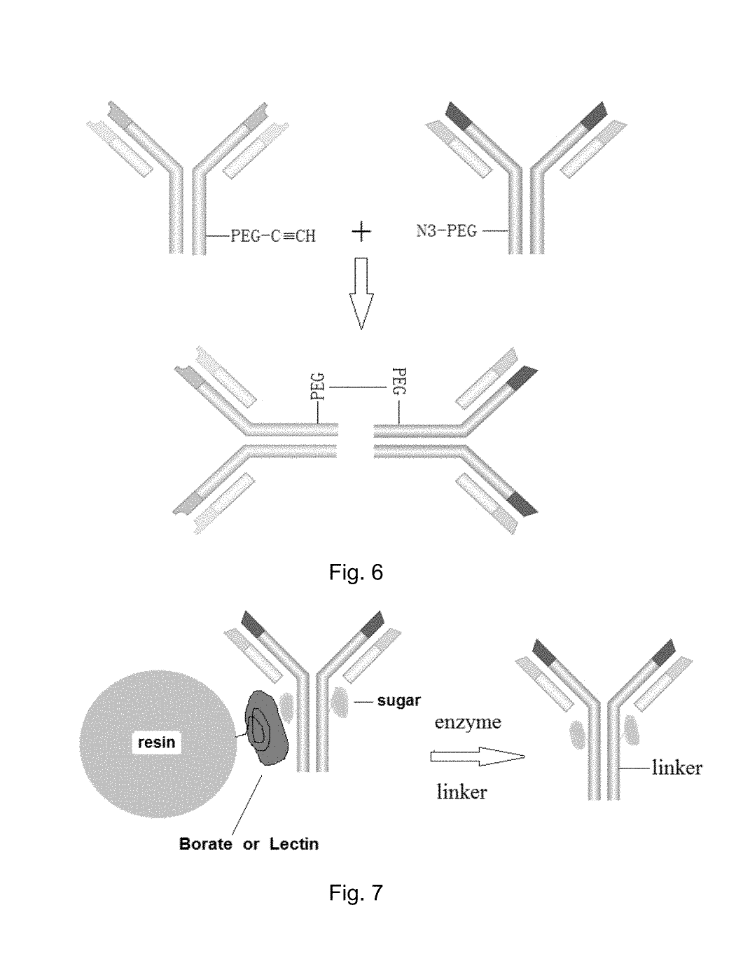

This design uses affinity group targeting the carbohydrate on the antibody to selectively protect one FC conjugation site on the antibody to achieve the mono conjugation. Suitable affinity resins include borate based affinity solid phase support or lectin based affinity phase support (FIG. 7). When one side of the antibody is protected, the other side can be selectively modified (e.g. site specific conjugation using enzyme such as mTGase).

Borate is a carbohydrate chelators and borate based column is widely used in separating carbohydrate, many are commercially available (e.g. from Sigma). Different borate also has different affinity to different sugar. Lectins are carbohydrate-binding proteins, most are from plant, which is used as antivirus/bacterial drug for animals. Different lectin has selectivity for different carbohydrate. Lectin column is also used in studying carbohydrate. Lectin or borate based resin can also be a useful tool for large scale purification of antibody drugs during ADC labeling. They can also be used for protein mono labeling other than antibody if the protein has carbohydrate modification.

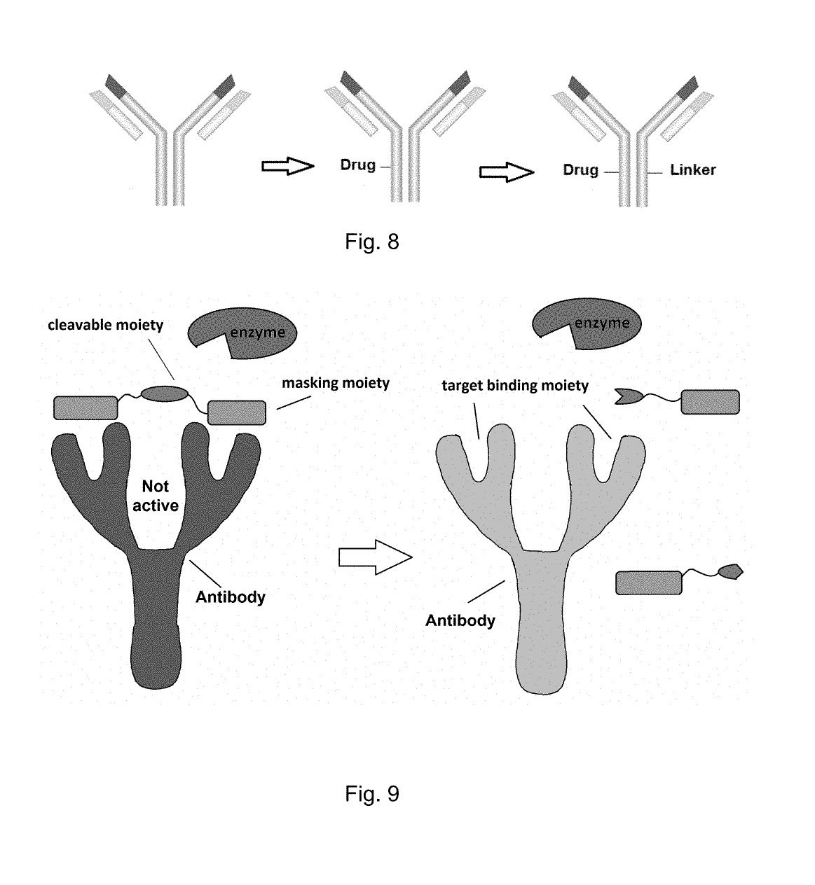

If mono labeling drug on the antibody can be done efficiently, then the later mono labeling of linker labeling can be done easily (FIG. 8).

Using ADC made of BsAb against two makers on the target cell will increase the specificity of drug delivery.

Bi Specific Antibody can be used for cytoplasm target. For example, in lupus, the key auto antibody causing the damage to the cells is the auto antibody against dsDNA. They are released from lysosome after internalization and bind with nucleus to cause cell damage. There are also many antibodies are against cytoplasm target. It is known that many cell surface receptors are reused after been internalized: suggesting it is not digested in lysosome.

Similarly, antibody against tublin can be used instead of MMAE or other toxin in the ADC. Therefore the ADC is essentially an antibody (e.g. for HER2)-antibody (e.g. for tubulin) conjugate, in another word, a bi-specific antibody. The advantage of using antibody instead of toxin as effector is that AB is much less toxic and can have high affinity and specificity, therefore less concern on side effect and toxicity due to potential release of toxin in blood circulation. Furthermore, the effector antibody may not need to target tubulin; it can be antibody against many other cytoplasm in tumor cells (e.g. tolemarase).

One issue with ADC for drug is that there are limited cell surface markers on cancers cells can be used for antibody and even HER2 is only positive in 30% patients. To expand the application of the above BS-Antibody strategy, the targets can be extended to diseases beyond cancer. There are many cytoplasm targets for many diseases and a lot of drugs are against cytoplasm targets, bi-specific antibody can be used as therapeutics against them: one AB against cytoplasm target and one against cell surface marker to help the effector AB uptaken by the cell.

The rate of internalization of antibody dimer should not be a big problem as size is not a key factor affecting internalization in many cases. A much bigger virus can be internalized easily. Even if it was a concern, monomer type Bs antibody or adding a positively charged linker can be used to improve internalization.

An antibody (against gp120)—toxin conjugate has been made to kill HIV virus infected T cell (HIV infected T cells express HIV gp 120 on T cell surface). This strategy can be applied to many other virus infections since the infected cell will express virus protein on their surface. However, toxin is toxic and has their limitations.

A more universal strategy is to use antibody-virus inhibitor conjugates instead. Many virus inhibitors are very potent and have suitable functional groups to be linked to antibody with very low toxicity to cells. For example, antibody against gp120 or CD3, CD4 can be conjugated to HIV RT inhibitor (e.g. AZT) or HIV protease inhibitor(e.g. Amprenavir) to treat HIV infection; antibody against CK18, CK19 or HBV surface antigen conjugate with RT inhibitor can be used to treat HBV infection.

A benefit of using virus inhibitor is that the antibody in ADC can target the normal cell surface marker (e.g. using ADC targeting CD3, 4 for T cell to treat HIV; using ADC targeting CK 18 for hepatic cell to treat HBV, HCV), which is prohibited for using toxin (will kill the normal cell) and the toxicity is very lower. It will also allow the inhibition of virus infecting cells before the virus protein is expressed on the host cell surface. There are applications for ADC in other diseases besides treating virus infection and cancer.

The current also invention discloses novel strategy for antibody or aptamer construction, which can be activated by enzyme, they are called self assembly probody and protamer respectively.

Probody (e.g. those developed by Cytomx) is antibody that can be activated (having binding affinity to antigen after activation) by enzyme. Protamer is aptamer that can be activated (having binding affinity to target after activation) by enzyme.

US patent/patent allocation U.S. Pat. No. 8,529,898, US 2010/0189651, US20130315906 and US20140010810 disclosed antibody construction called probody that can be activated by enzyme.

The probody in the prior art are activatable binding polypeptides (ABPs, e.g. antibody), which contain a target binding moiety (TBM), a masking moiety (MM), and a cleavable moiety (CM) are provided. Activatable antibody compositions, which contain a TBM containing an antigen binding domain (ABD), a MM and a CM are provided. Furthermore, ABPs which contain a first TBM, a second TBM and a CM are provided. The ABPs exhibit an “activatable” conformation such that at least one of the TBMs is less accessible to target when uncleaved than after cleavage of the CM in the presence of a cleaving agent (e.g. enzyme) capable of cleaving the CM. Further provided in the prior art are libraries of candidate ABPs, methods of screening to identify such ABPs, and methods of use. Further provided are ABPs having TBMs that bind VEGF, CTLA-4, or VCAM, ABPs having a first TBM that binds VEGF and a second TBM that binds FGF, as well as compositions and methods of use. The prior art disclosure provides modified antibodies which contain an antibody or antibody fragment (AB) modified with a masking moiety (MM). Such modified antibodies can be further coupled to a cleavable moiety (CM), resulting in activatable antibodies (AAs), wherein the CM is capable of being cleaved, reduced, photolysed, or otherwise modified. AAs can exhibit an activatable conformation such that the AB is more accessible to a target after, for example, removal of the MM by cleavage, reduction, or photolysis of the CM in the presence of an agent capable of cleaving, reducing, or photolysing the CM.

The current invention discloses novel probody format. In the prior art, the masking moiety MM is covalently conjugated to the target binding moiety TBM (e.g. antibody, receptor, ligand for receptor such as VEGF). In the current invention, the difference is that the masking moiety MM is not covalently linked to the TBM (e.g. antibody, receptor, ligand for receptor such as VEGF). The cleavable moiety (CM) connect two MM instead of connecting the MM with the TBM in the prior art. Optionally a linker/spacer (e.g. a peptide or PEG) can be added between the MM and CM to allow optimal binding of two MM to the two Fab sites (or other binding moieties such as VEGF). The TMB such as antibody, MM and CM sequence can be essentially the same as these in the prior art disclosure except the linking between them is different as described above. The tandem MM strategy in the prior art can also be applied (FIG. 9). The probody in the current invention is a bound complex instead of a single molecule as that in the prior art. This strategy allows the use of the current available antibody or protein without the need to develop a new conjugate, therefore simplify the drug development process. The enzyme will cleave the CM and activate the TBM by exposing the previously blocked binding sites. One can either use the pre formed complex or give the patient the two component separately to allow the complex form in vivo.

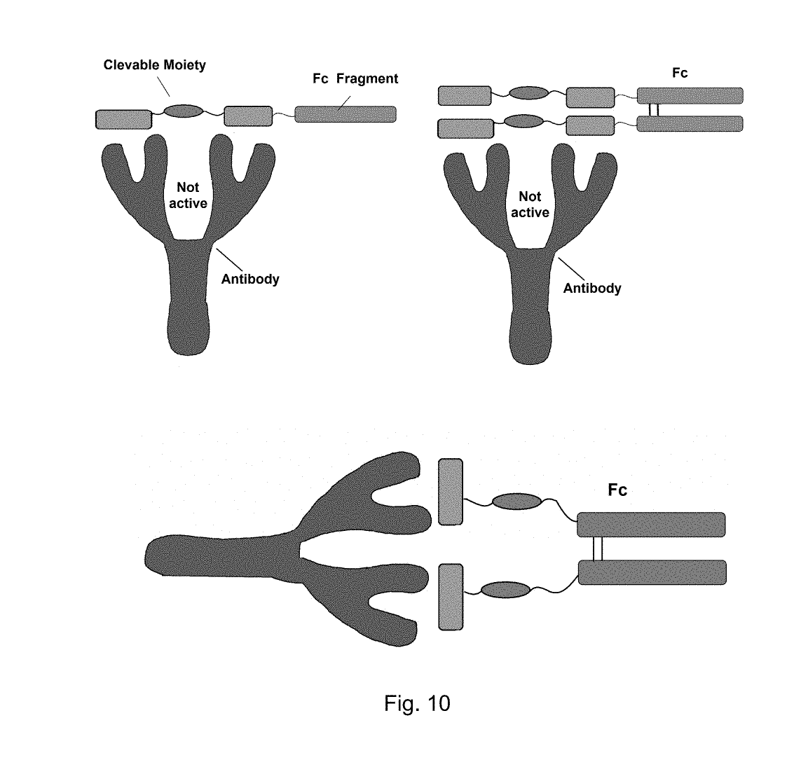

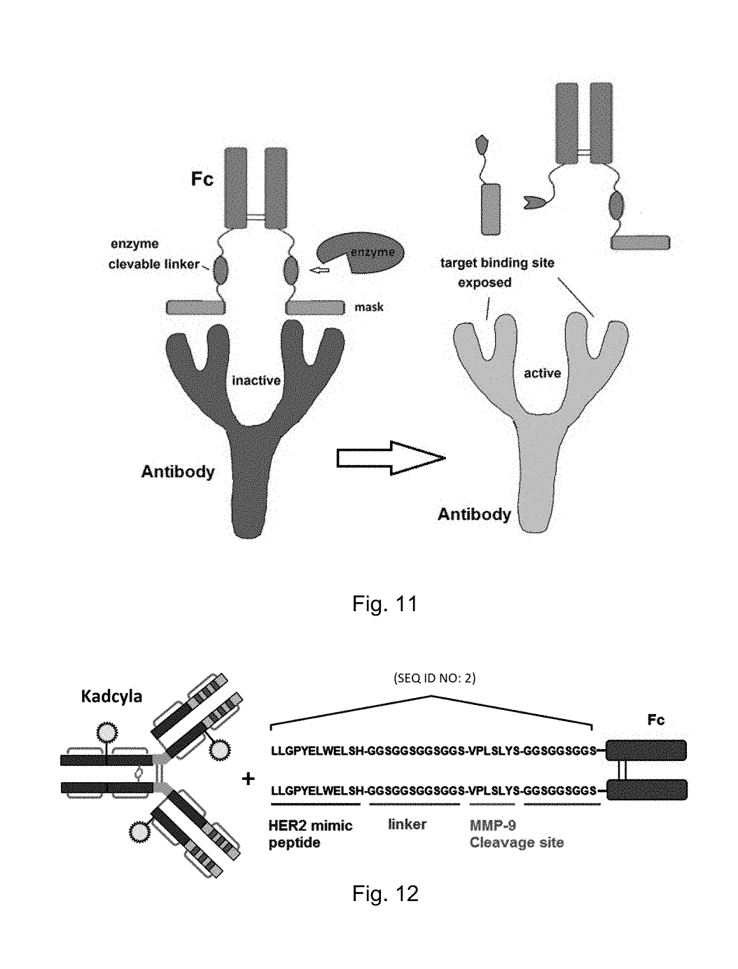

Preferably antibody Fc or its fragment (e.g. single chain) can be connected to the MM (either by chemical conjugation or fusion/expression) to increase its half life (examples see figure below). Besides Fc tag, other half life extender (e.g. PEG, albumin, lipophilic tag, Xten, carboxyl-terminal peptide (CTP) of human chorionic gonadotropin (hCG)-beta-subunit) currently used to extend in vivo protein half life can also be attached to the MM covalently to reduce its in vivo inactivation/elimination (FIG. 10-11). In some embodiments the antibody can be engineered that the binding of ligand (masking moiety) with antibody does not activate complement. The antibody can have mutations that preclude binding to FcγR and/or C1q. The antibody (or other TBM) can be conjugated with drugs as a targeted drug delivery system. Excess amount of cleavable moiety (CM)—MM conjugate can be used to inhibit the antibody (or other TBM) binding completely.

In one example (FIG. 12), Trastuzumab emtansine self-assembly probody is disclosed. LLGPYELWELSHGGSGGSGGSGGSVPLSLYSGGSGGSGGS(SEQ ID NO: 2) containing a HER2 mimic peptide, linker peptides and MMP-9 substrate peptide is fused with Fc, which forms a self assembly complex with Trastuzumab emtansine to block its binding affinity with HER-2 when no MMP-9 is present. The matrix metallopeptidase 9 (MMP-9) cleave the Fc-Mask peptide; release the active Trastuzumab emtansine (Kadcyla) to bind with HER2 on the tumor cell for targeted cancer therapy.

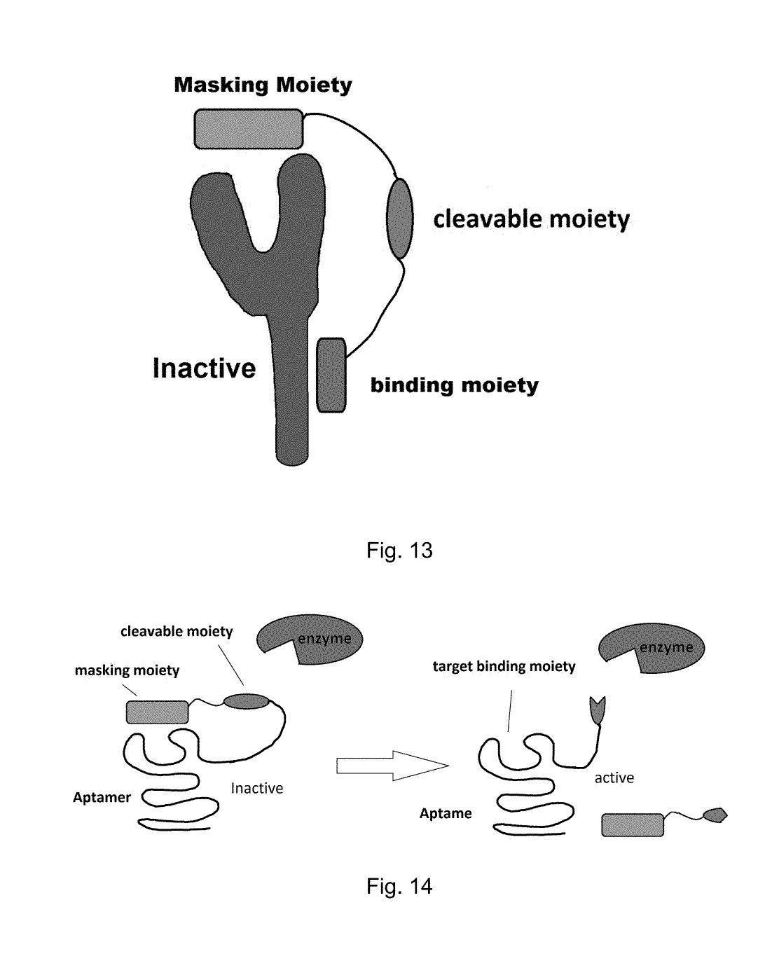

The two MM can also be heterogenic. One binds with the active site of the protein (e.g. the Fab or binding part of the protein), another binds with another part of the protein (non-TBM binding/active site). In this scenario, sometimes one of the MM is not a masking moiety anymore; it is essentially a binding moiety (FIG. 13). In FIG. 13, the masking moiety is a binding ligand for TBM while the binding moiety is protein A that binds with the Fc of the antibody.

The current invention also discloses novel protamer that can be activated by enzyme to restore its binding affinity. It is similar to probody except the activatable binding polypeptides (e.g. antibody) is replaced by an aptamer. The designs of protamer are illustrated in the FIG. 14. In one format, the aptamer is conjugated with a CM and then a MM covalently. The sequence of the CM can be the same as those used in probody. The MM is an affinity ligand (e.g a peptide that can bind with the aptamer binding domain or a complementary nucleic acid sequence) to the aptamer that can block the binding affinity of the aptamer. When the activating enzyme (or other condition such as low pH or recuing environment or light) is not present, the target binding affinity of the protamer is blocked by the masking moiety. When the enzyme is present, the enzyme will cleave the CM and activate the aptamer by exposing the previously blocked aptamer binding site.

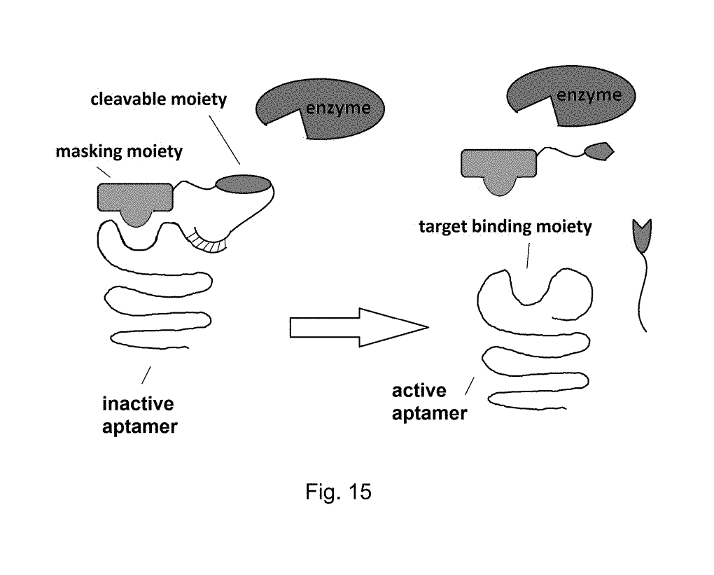

Alternatively, the CM can also be linked to the aptamer non-covalently, similar to the novel probody described in the current invention. For example (FIG. 15), the CM is linked to a nucleic acid sequence that can bind with the aptamer, therefore bind with the aptamer non-covalently.

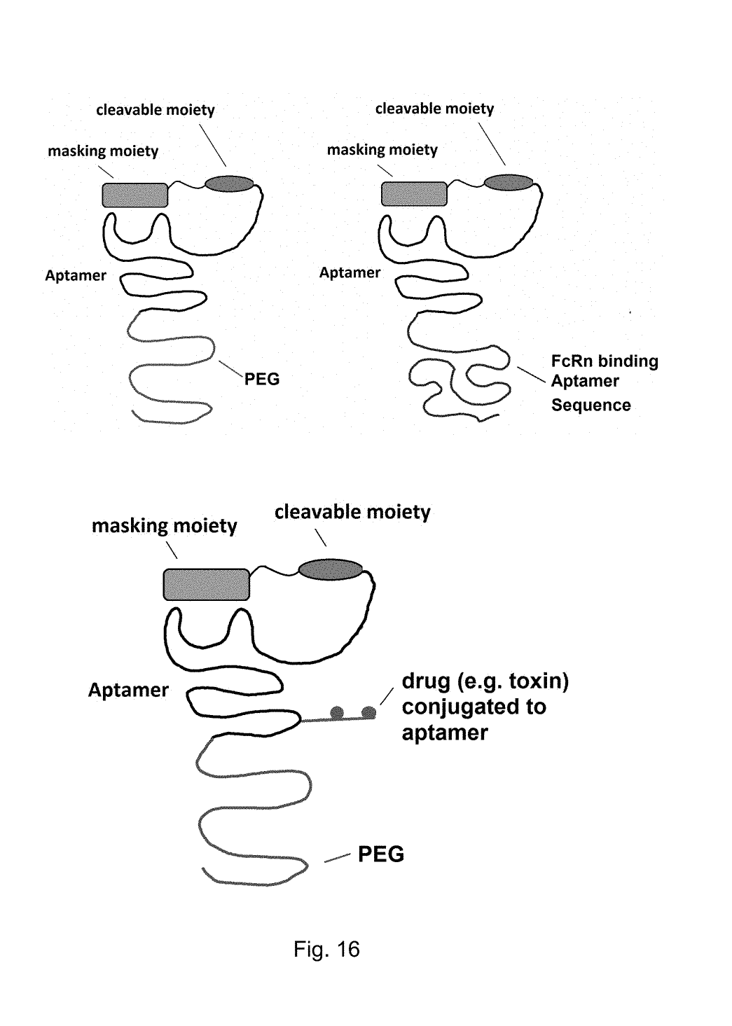

The aptamer can also be conjugated with a drug (e.g. toxin, radioactive element, chelater-radioactive element complex) to act as a targeted drug delivery system similar to the antibody drug conjugate. The aptamer can also be conjugated with a PEG or Fc domain or other polymer (e.g. Xten from Amunix) or tag (e.g. an affinity tag that can bind with albumin) to extend its in vivo half life. The aptamer can also have a binding sequence (made of another nucleic acid sequence) mimic the Fc domain of antibody to allow the recycle of the aptamer. This sequence is essentially an aptamer that mimic the function of Fc domain that can bind with FcRn at acidic pH of (<6.5) but not at neutral or higher pH. Examples are shown in the FIG. 16.

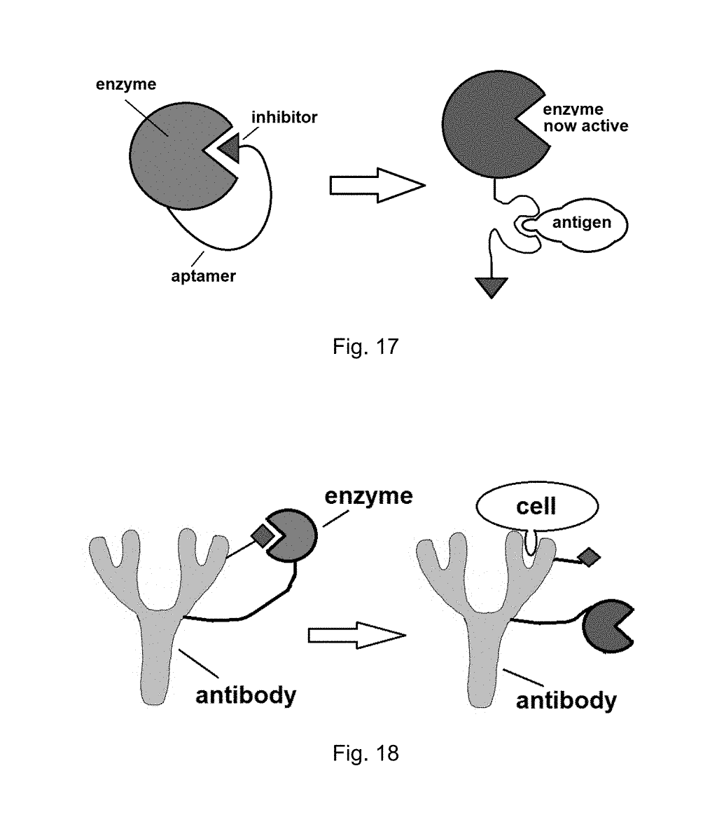

The current invention discloses novel strategy for enzyme construction which is called Binding Based Prozyme. Binding Based Prozyme is enzyme conjugated with affinity ligand (e.g. aptamer or antibody). When its affinity ligand does not bind with the target, the enzyme has low or no activity. When it binds with the target, the enzyme is activated to show high catalytic activity (FIG. 17). The affinity ligand is covalently coupled to the enzyme; the affinity ligand is also coupled with an enzyme inhibitor (e.g. a molecule that can mask the enzyme catalytic center) or a molecule that can block the enzyme's active site. When the target molecule (antigen) is not present, the enzyme inhibitor binds with the enzyme to block the enzyme's activity. When the target molecule (antigen) is present, the aptamer bind with the antigen and the conformation change of the aptamer due to binding inhibits the binding of the enzyme inhibitor with the enzyme, therefore exposes the active enzyme catalytic site and restores the enzyme activity.

In one example, glutathione S-transferase-PEG 20-CGA GAG GTT GGT GTG GTT GG (SEQ ID NO: 3)-fluorescein-3′ is made by coupling 5′-PEG 20-CGA GAG GTT GGT GTG GTT GG (SEQ ID NO: 3)-fluorescein-3′ having a —COOH group at the PEG end with the amine group on the enzyme using EDC. -CGA GAG GTT GGT GTG GTT GG (SEQ ID NO: 3)—is a thrombin-binding DNA aptamer. Fluorescein is an inhibitor of glutathione S-transferase. The resulting conjugate has low enzyme activity when there is no thrombin and has high enzyme activity when thrombin is present.

The FIG. 18 shows the resulting steric hindrance from binding of antibody with the antigen releases the active enzyme from its inhibitor therefore restores the enzyme activity. The enzyme inhibitor is conjugated close to the antibody's antigen binding site and the enzyme is conjugated to the antibody with a linker. When the antigen is not present, the enzyme is blocked by the inhibitor. When the antigen is present, the antibody will bind with the antigen and the resulting steric hindrance from binding of antibody with the antigen prevents the binding of the inhibitor with the enzyme, therefore restore the activity of the enzyme.

This strategy can be used to provide therapeutic enzyme conjugate that become activated enzyme when it binds with certain target, therefore provides better target specificity. For example, the affinity ligand can bind with certain cell or pathogen surface marker and the enzyme can produce certain biological effect to the cell or pathogen. When there is no target cell/pathogen present, the enzyme is inactive, when the maker bearing cell/pathogen is present, the enzyme conjugate bind with the cell/pathogen and the enzyme become active, produce therapeutical effect to the cell or pathogen. In one example, the affinity ligand is an aptamer or antibody against HER2, the enzyme is a protease or an enzyme that can convert an anti caner prodrug to its active form. This Prozyme can be used to selectively inactivate the HER2 positive cancer cells. In another example, the affinity ligand is an aptamer or antibody against gp-120, the enzyme is a hydrolase that can damage the virus particle. This Prozyme can be used to selectively inactivate HIV virus.

Alternatively, the affinity ligand can bind with one part of the target macromolecule (or its complex) and the active enzyme can act on the other part of the macromolecule (or its complex), when the target macromolecule (or its complex) is present, the enzyme will be active and act on the target macromolecule (or its complex). In one example, the target is amyloid plaques. The affinity ligand can bind with amyloid plaque and the enzyme is a hydrolase that can cleave peptide bonds. This Prozyme can be used to hydrolyze amyloid plaques. This method also provides a new method to develop new enzyme, by coupling a specific ligand to enzyme that has a broad substrate spectrum. The resulting enzyme will have higher selectivity: only act on the target that can bind with the affinity ligand.

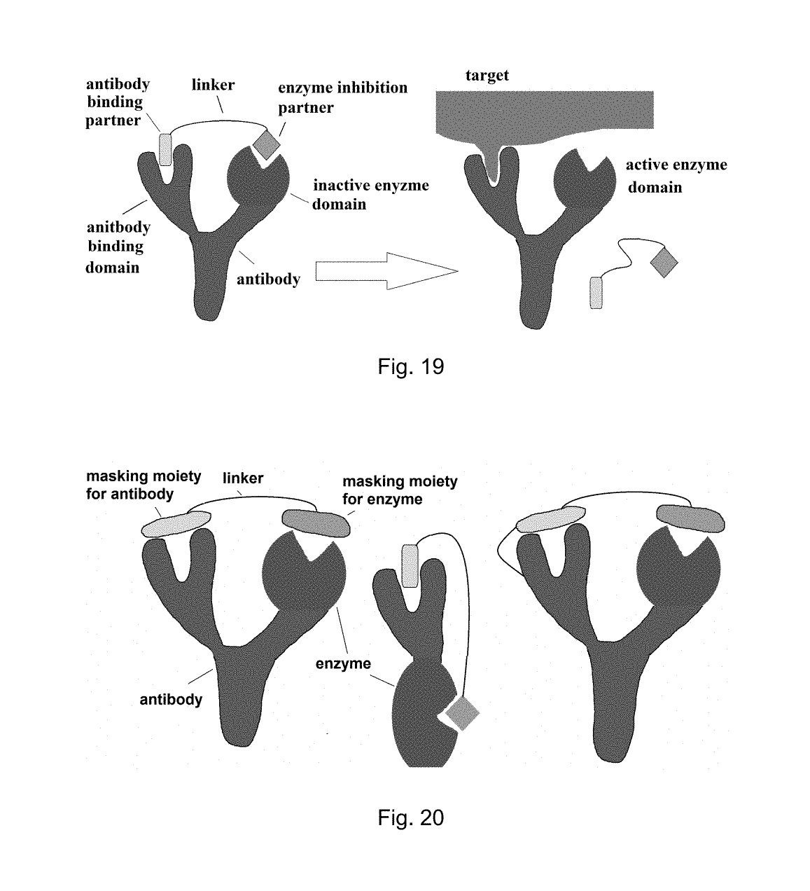

Another format (FIG. 19) is to use an ABP (antibody binding partner)-linker-EIP (enzyme inhibition partner) to form a non-covalent complex with the antibody-enzyme fusion protein, in which the enzyme domain is inactivated by the EIP. The ABP can be the antigen or MM used in the probody. The EIP can be an enzyme inhibitor or a masking molecule that mask the enzyme active center. The linker length is optimized to ensure the maximal binding of ABP and EIP to the fusion protein. When the antibody binding target is present, the ABP-linker-EIP is displaced and the enzyme activity is restored. ABP-linker-EIP can be added in excess amount to inhibit the enzyme activity to the desired level when binding target is not present. In some embodiments, the ABP can also be conjugated to the antibody, which will result in a covalent complex with the antibody-enzyme fusion protein. Examples of possible formats are shown in the FIG. 20. Besides antibody or antibody fragment, other affinity ligand for the target such as aptamer can also be used to conjugate/fuse with the enzyme.

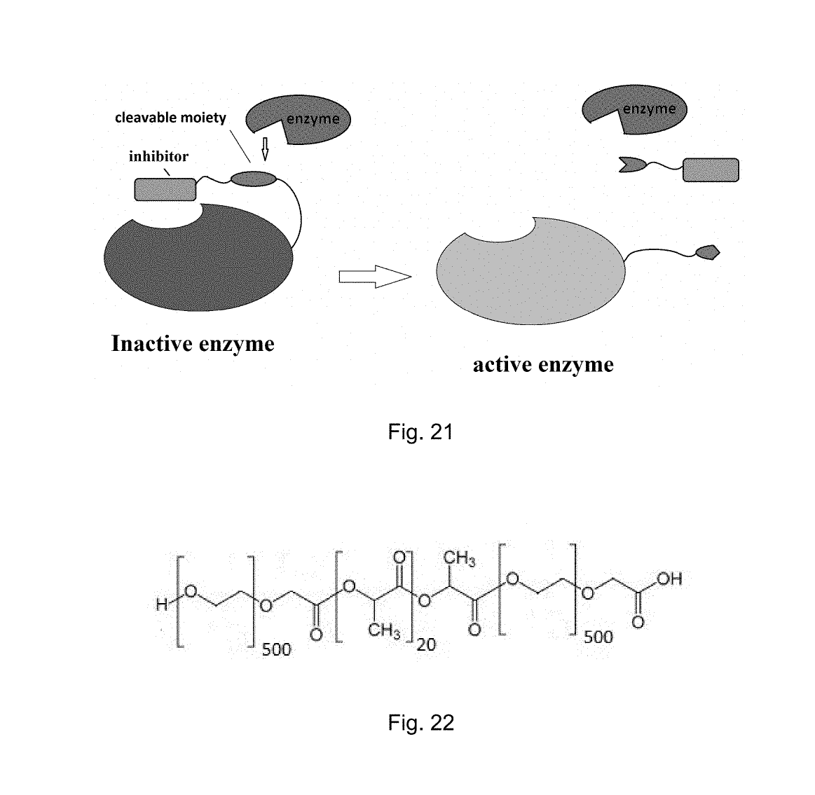

The current invention discloses novel strategy for enzyme construction which is called Cleavage Based Prozyme. Cleavage Based Prozyme is an activatable enzyme conjugated with enzyme inhibitor via a second enzyme (or other condition such as low pH or reducing environment) cleavable moiety, a mechanism similar to probody. When there is no second enzyme or suitable cleavage condition, the enzyme has low or no activity. When there is second enzyme or other cleavage condition, the enzyme is activated to show high catalytic activity (FIG. 21). The second enzyme can be either the same as the activatable enzyme or an enzyme with different catalytic activity.

The cleavable moiety is covalently coupled to the enzyme; the cleavable moiety is also coupled with an enzyme inhibitor (e.g. a molecule that can mask the enzyme catalytic center). In one example, glutathione S-transferase-PEG 20-CCCCAAA-fluorescein-3′ is made by coupling 5′-PEG 20-CCCCAAA-fluorescein-3′ having a —COOH group at the PEG end with the amine group on the enzyme using EDC. -CCCCAAA is DNA fragment which can be cleaved by DNase. Fluorescein is an inhibitor of glutathione S-transferase. The resulting conjugate has low enzyme activity when there is no DNase and has high enzyme activity when DNase is present.

This strategy can be used to provide therapeutic enzyme conjugate that become activated enzyme when it is close to a target having the second enzyme, therefore provides better target specificity. For example, the second enzyme can be on the surface of or inside certain cell or pathogen and the enzyme can produce certain biological effect to the cell or pathogen. When there is no target cell/pathogen present, the enzyme is inactive, when the second enzyme bearing cell/pathogen is present, the enzyme conjugate will be cleaved by the cell/pathogen and the enzyme become active, produce therapeutical effect to the cell or pathogen. In one example, the cleavable moiety is a special peptide sequence that can be cleaved by a protease, the enzyme is an esterase that can convert an anti caner prodrug to its active form. This prozyme can be used to selectively inactivate the said protease rich cancer cells.

Furthermore, the prozyme can be conjugated to or fused to an affinity ligand (e.g. an antibody) to provide further selectivity. In one example, the antibody is an antibody against HER2, therefore the Prozyme-antibody conjugate can be used to kill HER-2 positive cancer cells. In one example, the cleavable moiety and the linker connecting antibody with the enzyme (e.g. those currently used in ADC drugs) are substrate of the enzyme in lysosome. After endocytosis, the prozyme-antibody conjugate in the lysosome is cleaved to release the active enzyme to kill the cancer cell. Hydrophilic carbon chain can be introduced into the conjugate to help breaking the lysosome membrane.

The prozyme can also be used a signal amplification system (e.g. for ELISA). For example, the activatable enzyme can be a HRP and the second enzyme can be a DNase. The detection antibody in ELISA is conjugated with DNase. The substrate solution will contain prozyme and HRP substrate for signal amplification.

The current invention discloses a method and formulation combination to improve the in vivo half life and potency of biological active protein by combining protein with protein-antibody immuno complex and administrating it to the patient, in which the amount of protein is greater than the binding capacity of antibody to provide free unbound protein in the formulation. The current invention also discloses biological active protein that can be used as potential drug in oligomer format (e.g. trimer format, which connects 3 proteins with either cleavable or non cleavable linkers) and its application in HGH oligomer (e.g. trimer) to increase their in vivo half life and potency.

Modification of proteins with hydrophilic polymers is an effective strategy for regulation of protein pharmacokinetics. However, conjugates of slowly or non-biodegradable materials, such as poly ethylene glycol (PEG), are known to cause long-lasting cell vacuolization when its MW is high, in particular in renal epithelium. Conjugates of more degradable polymers, e.g., polysaccharides, have a significant risk of immunotoxicity. Polymers that combine complete degradability, long circulation in vivo, and low immuno and chemical toxicity would be most beneficial as protein conjugate components. In one aspect the current invention uses biodegradable linker to connect PEG block polymer (or other synthetic polymer) to generate large MW biodegradable PEG (or other synthetic polymer). The resulting big MW PEG (or other synthetic polymer) can break into small PEG (or other synthetic polymer) to increase drug potency/PEG (or other synthetic polymer) clearance and reduce toxicity of large PEG (or other synthetic polymer). Proteins with MW<70 K can be rapidly cleared by kidney. People use PEG to conjugate to proteins to increase its MW to reduce the kidney clearance rate. However large PEG (MW>40K) can cause kidney damage and has high viscosity which makes protein drug injection difficult. Examples of biodegradable linker include peptide, ester, polylactic acid, carbohydrate, polyal(e.g. those in U.S. Pat. No. 8,524,214), biodegradable hydrophilic polyacetal, poly (1-hydroxymethylethylene hydroxymethylformal, polyphosphate, Mersana's Fleximer® polymer and etc. Peptide that can be cleaved with endogenous peptidase/protease and those cleavable linkers used in ADC (e.g. hydrazone linker,disulfide linker, peptide linker such as -Val-Cit-) can also be used to connect small PEG fragment/blocks (or other synthetic polymer), which can undergo enzyme cleavage, acidic (e.g. proton-catalyzed hydrolysis at lysosomal pH), proteolytic or redox cleavage.

When PEG is used It has the following general structure: (PEG-biodegradable linker)_{N}-protein (N is an integer). Optionally there is a attachment moiety (e.g. a chemical bond or conjugation linker) between the (PEG-biodegradable linker)_{N} and the protein to connect them together. One example is given in the FIG. 22, which is a block polymer made of two PEG blocks connected with a biodegradable polylactic acid. One end of the PEG has a —COOH group, which can be used to couple to the amine group of the lysine on the protein surface. Other synthetic polymer such as poly vinyl alcohol can also be instead of PEG.

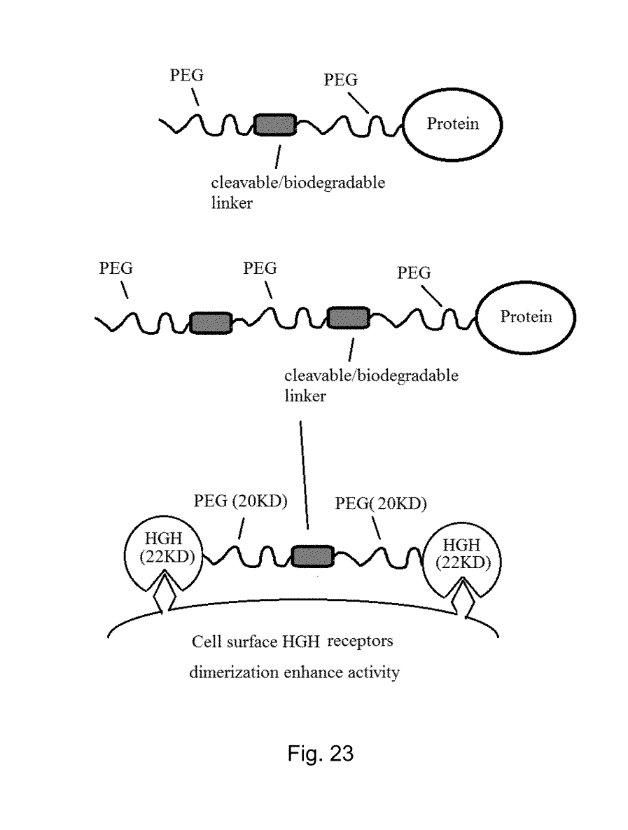

In another example HGH dimer is constructed. Human growth hormone (HGH, MW=22K) needs daily injection due to its fast kidney clearance. Biodegradable HGH dimer can be used as a better alternative: HGH-PEG(20K)-cleavable linker-PEG(20K)-HGH MW=85K>70K (MW cutoff for kidney clearance). In one embodiment the PEG has an amine terminal, which can couple to the Gln on the HGH by mTgase. The FIG. 23 illustrates different formats of biodegradable PEG and the biodegradable HGH dimer.

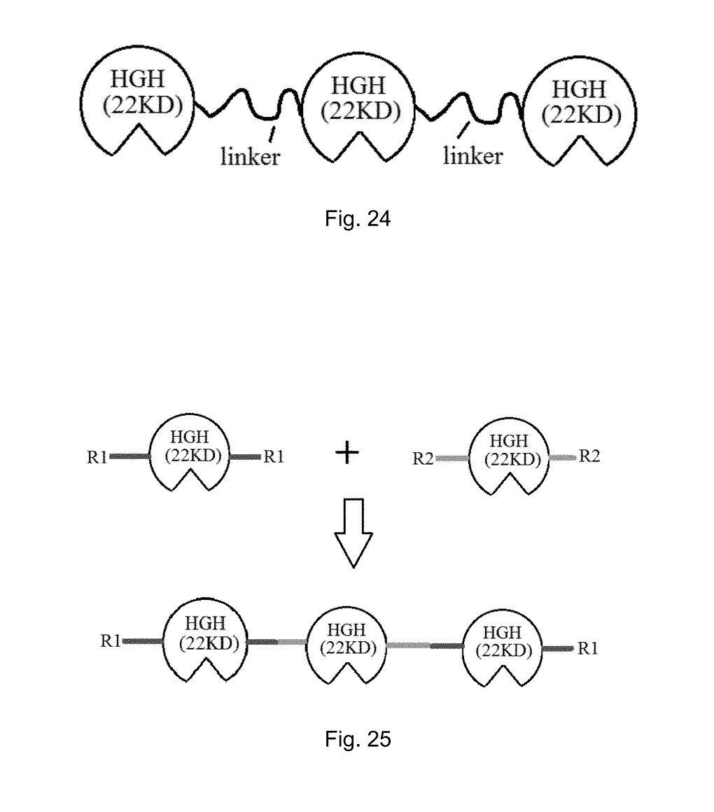

Alternatively, 3 proteins can be covalently connected to form a trimer with two linkers, which will further increase its size and molecular weight therefore extend its half life in vivo. The linker can be either biodegradable or non biodegradable. Preferably the molecular of the resulting trimer is greater than 60 KD. In some embodiments it is greater than 70 KD. The preferred linker should have a preferred molecular weight that make the total trimer >60 KD. The linker can be PEG, peptide or other biologically acceptable linker. FIG. 24 shows an example of HGH trimer which can extend HGH in vivo half life.

The two linkers connecting the 3 HGH can be the same. For example, it can be a PEG or a hydrophilic peptide (e.g. peptide rich of Ser, Thr, Glu, Asp) having a MW between 500˜15 KD.

FIG. 25 shows another example of the HGH trimer and its preparation. Each HGH has two modifications resulting in two reactive groups. R1-PEG-NH2 and R2-PEG-NH2 can be site specifically conjugated to HGH separately by MTgase. R1 and R2 are reactive groups (e.g. those in click chemistry, —SH/maleimide pair and etc) that can conjugate together specifically to form a covalent bond. Next the resulting two HGH are mixed and the covalent bond is formed connecting R1 and R2. To ensure the trimer is the main product other than tetramer and polymer in higher degree, HGH with R1 can be added in excess (e.g. 10 folds more), or one of the R1 can be protected/blocked before the coupling.

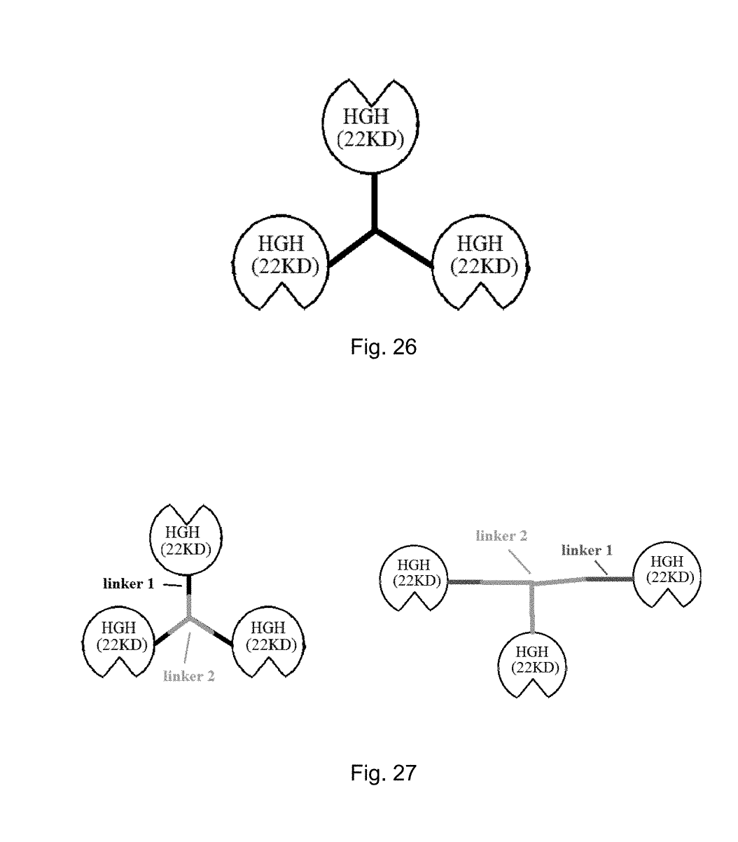

The trimer can also be constructed with a linker having three arms as shown in FIG. 26. For example, the 3 arm linker can be a three arm PEG or a three arm hydrophilic peptide (e.g. peptide rich of Ser, Thr, Glu, Asp) or their conjugate having a MW between 2K˜20 KD. In another example (FIG. 27), linker 1 and liker 2 are connected covalently. Linker 2 and linker 1 are conjugated to HGH (to its Gln) with MTgase and then coupled together using the reactive group on linker 1 and liker 2. Linker 1 and 2 can be functionalized PEG having a MW between 500˜10 KD.

Alternatively, extended in vivo half life of pharmaceutically active protein can be achieved by cross linking the protein non-covalently with linker having multiple affinity group (e.g. antibody or its fragment such as Fab, aptamer or an affinity peptide that can be generated using phase display or the method similar to the development of masking peptide used in probody or screening or rational design) for the protein. Optionally the linker is biodegradable (e.g. an enzyme cleavable peptide). The affinity group can bind with the protein at its active site or non active site.

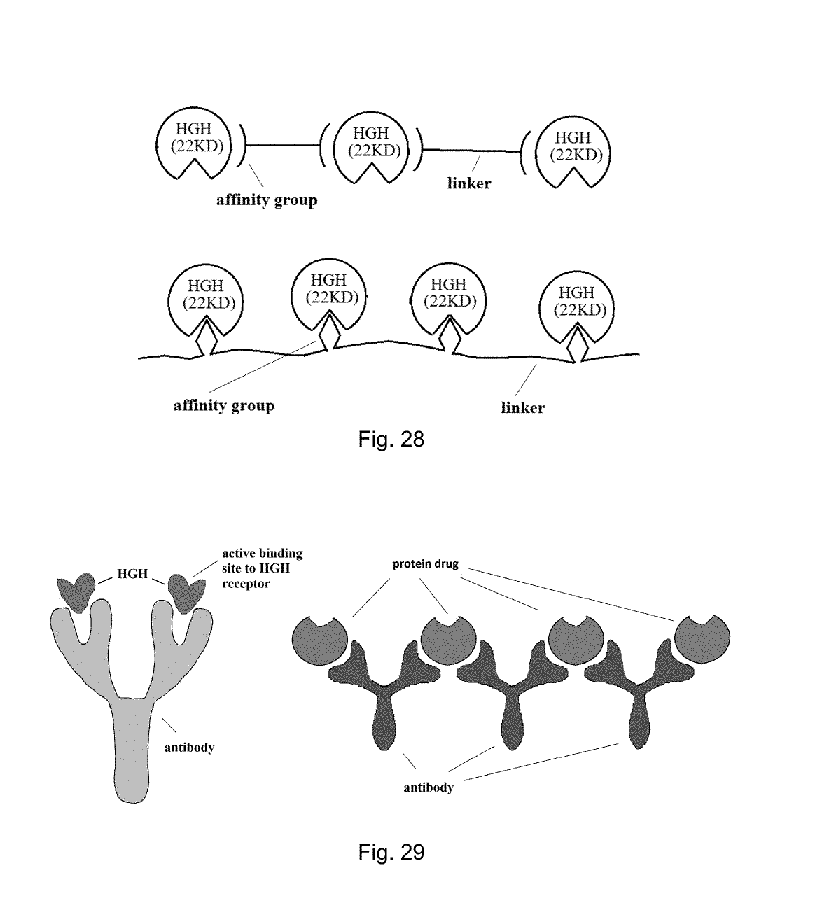

FIG. 28 illustrates two formats to crosslink HGH to extend its in vivo half life. One format is to use a linker having affinity groups binding to HGH's non receptor binding site at both ends to crosslink HGH. In one example, the affinity group is a 30 AA (amino acid) peptide and the linker is a peptide having 10 AA or a short PEG. Another format is to have a linker carrying multiple affinity groups binding to HGH's receptor binding site.

The linker having multiple affinity groups can be a protein or a peptide having multiple affinity groups, e.g. an antibody, since each antibody has two binding sites. The binding site for the affinity groups can also be introduced artificially to the pharmaceutically active protein. For example, biotins can be attached to the target protein by expression or chemical conjugation and avidin can be used to crosslink the said biotinylated protein for longer in vivo half life. In some examples, the protein is modified with Thermo Scientific EZ-Link™ Sulfo-NHS-Biotinylation Kit (#21425) or EZ-Link™ Pentylamine-Biotin (#21345) using the provided protocol from the vendor and then dialyzed to remove the uncoupled. Next avidin or streptavidin is added to the biotinylated protein at 1:2 ratio in PBS for 30min to form the binding complex, which will have longer in vivo half life compared with the original protein.

Another format is to use protein specific antibody or antibody fragments or aptamer to form an immuno complex or aptamer-protein complex, which will have higher molecular weight (may also protect the protein from enzyme degradation) therefore slower elimination. The binding of antibody/aptamer can be either targeting the protein's active site or non active site. In one example, antibody against HGH's non binding region is mixed with HGH at 1:2 ratio to form its immuno complex, this complex can be used as therapeutics having extended half life to be administrated to the patient. It can also be two antibodies binding with one protein format (the sandwich type binding format similar to those seen in ELISA). Optionally the protein binding with antibody does not activate complement, which can be archived by engineering the antibody. Mutation can be introduced to the antibody FC to remove complement binding (e.g. to clq), binding to FcγR as well as binding to CR1. FIG. 29 shows two examples using the strategy described above. Bispecific antibody that binds to two different epitopes of the target protein can be used to crosslink the protein.

Alternatively, two antibodies targeting two different epitopes can be connected together (e.g. by fusion or conjugation) to act as a bispecific antibody to cross link target proteins. One example of this kind of two antibody conjugate is shown in FIG. 5 and FIG. 6. Antibodies or antibody fragments targeting different epitope of the protein (e.g. HGH) can be screened to obtain the antibody/antibody fragment providing the best potency and pharmacokinetic property (e.g. in vivo half life).

In some embodiments, antibody fragment containing the epitope binding region is used to form the immuno complex to extend the half life of protein. Suitable antibody fragment can be selected from F(ab′)2 (110 KD), Fab′ (55 KD) Fab (50 KD) Fv (25 KD) which can be cross-linked to improve its stability, scFV, di-scFV, sdAb or the like. In one example, Fab or half-IgG (rIgG) against HGH can be mixed with HGH at 1:1 ratio to form the immuno complex, which can be used as a controlled release HGH drug. Different Fab (e.g. Fab bind with different region of HGH) can be screened to achieve the desired in vivo stability. The resulting binding complex has a MW>70K therefore the kidney clearance rate is reduced. The MW of Fab (50K) ensures that it will have similar clearance rate as HGH therefore reduce the buildup of Fab against HGH.

Optionally the antibody or antibody fragment including FC fusion protein used in the current application can engineered/mutated on the FC to remove complement binding (e.g. to clq), binding to FcγR as well as binding to CR1. The Fc region can also be engineered/mutated to adjust its FcRN binding capability (e.g. provide higher binding affinity for longer Fc containing protein in vivo half life).

The current invention disclose methods for Protein drug half-life extension with Protein Drug Trimer (or higher degree oligomer) using protein as monomer building block. Many small therapeutic proteins (e.g. 10-30 KD) require high MW PEG to reduce rapid renal clearance (>60 KD). High MW PEG may cause cell vacuolation, reduced protein activity, solubility issues and high viscosity; and mono-PEGylation may not provide enough protection against protease/peptidase. The current invention discloses Protein Trimerization (or higher degree oligomer) for half life extension.

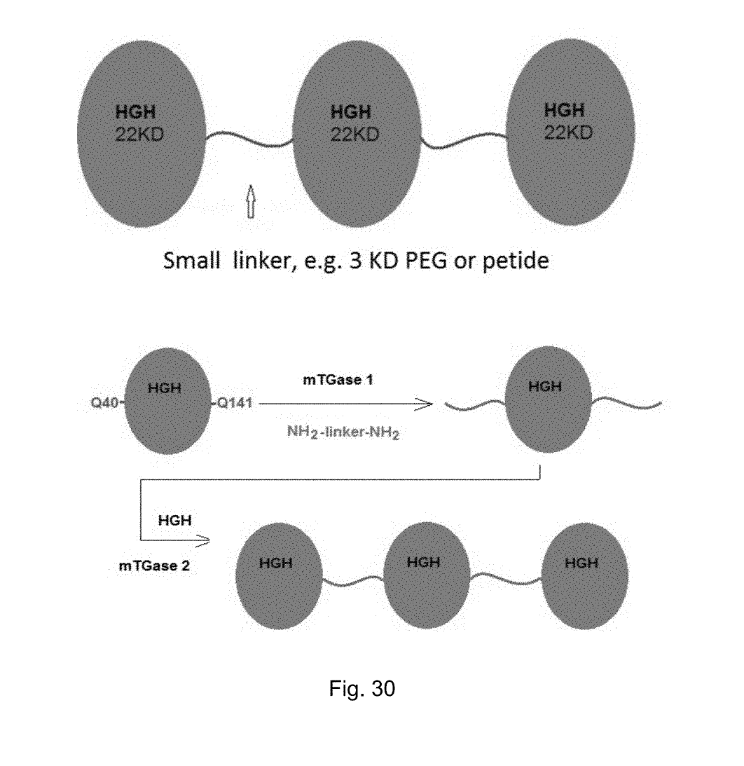

FIG. 30 shows examples of PEGylated HGH (Human Growth hormone) trimer for half-life extension using a small size PEG (or peptide) as linker and an example of its synthesis. The HGH suitable for the current invention can be HGH (Somatropin) from pituitary origin (191 amino acids, the SEQ ID No.1 disclosed in U.S. Pat. No. 8,841,249) having Accession Number: DB00052 (BIOD00086, BTD00086). For example, a low MW PEG (e.g. its MW can be a number between 5K˜20K) having —NH2 groups at its two ends can be used as a linker, alternatively, a peptide having 30˜200 amino acid residuals and two —NH2 groups at it two ends can also be used. The conjugation can be performed using transglutaminase (TGase) to couple the linker to the glutamine in the HGH. Preferably, the linker is introduced at the positions corresponding to positions glutamine 40 and/or glutamine 141 in HGH. The use of transglutaminase (TGase), and in particular microbial transglutaminase (mTGase) from Streptoverticillium mobaraenae or Streptomyces lydicus allows a selective introduction of the linker at positions 40 and/or 141, and the remaining 11 glutamine residues are left untouched despite the fact that glutamine is a substrate for transglutaminase. The protocol of MTGase can be found in many publications such as U.S. Pat. No. 8,841,249 and can be readily adopted for the current application. In the example shown in FIG. 30, excess linker (e.g. di-amino PEG at 10˜20 folds excess to the HGH amount) is added to the HGH and the coupling is performed with mTGase. The resulting HGH carrying two linkers on each HGH monomer is purified to remove unconjugated linker and unconjugated/mono conjugated HGH. Next excess amount of unconjugated HGH (e.g. 20 folds excess) is mixed with the previously prepared di-conjugated HGH and the coupling is performed with mTGase. The resulting conjugate is the HGH trimer having two linkers in the middle HGH and one linker on each end HGH. Using special mTgase can allow the site specific conjugation at either glutamine 40 or 141 or both.

For example, the use of a transglutaminase to attach PEG to HGH on glutamine residues has previously been described in U.S. Ser. No. 13/318,865 and U.S. Ser. No. 12/527,451. The method may be used in accordance with the present invention for attachment of the linker and linker conjugated with HGH. The TGase used can be microbial transglutaminase according to U.S. Pat. No. 5,156,956. In one embodiment, a hGH is dissolved in triethanol amine buffer (20 mM, pH 8.5, 40% v/v ethylene glycol). This solution is mixed with a solution of amine donor linker, e.g. NH2-PEG-NH2 dissolved in triethanol amine buffer (200 mM, pH 8.5, 40% v/v ethylene glycol, pH adjusted to 8.6 with dilute hydrochloric acid after dissolution of the amine donor). Finally a solution of mTGase (˜0.5-7 mg/g hGH) dissolved in 20 mM PB, pH 6.0 is added and the volume is adjusted to reach 5-15 mg/ml hGH (20 mM, pH 8.5). The combined mixtures are incubated for 1-25 hours at room temperature. The reaction mixture is monitored with by CIE HPLC. The resulting HGH having two linkers on each protein is purified.

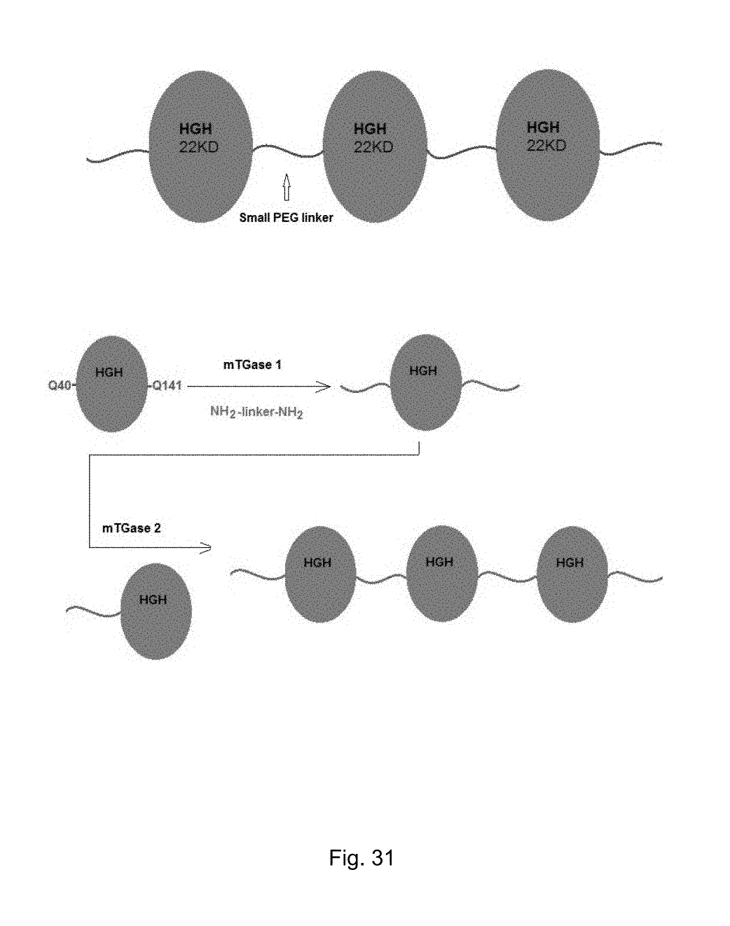

Alternatively, if excess amount of mono-conjugated HGH (e.g. 20 folds excess) is mixed with the previously prepared di-conjugated HGH and the coupling is performed with mTGase. The resulting conjugate is the HGH trimer with two linkers on all HGH (FIG. 31). In some embodiment, the linker for preparing the mono-conjugated HGH has one end with —NH2 group and another end without —NH2 group. By using special mTgase having different substrate specificity and altering the conjugation sequence and ratio, different trimer or oligomer can be prepared readily by skilled in the art.

Other site specific conjugation method can also be used to construct the oligomer. It could be as chemo selective synthesis such as click chemistry, thiol maleimide coupling and etc. It can also be enzyme based coupling other than mTgase conjugation, such as sortases based conjugation as well as the combination of different conjugation method. Sortase, particularly sortase A from S. aureus, has been recognized for some time as a useful protein engineering tool, allowing the ligation of oligo-glycine-containing polypeptides or small molecules to proteins containing a sortase-penta-peptide motif (LPXTG (SEQ ID NO: 6) in case of S. aureus sortase A, LPXTG : Leu-Pro-any-Thr-Gly (SEQ ID NO: 6)), e.g.: RLPXTG (SEQ ID NO: 7)+GGGGG (SEQ ID NO: 8)->LPXTGGGGG (SEQ ID NO: 9). The protocol of sortase based conjugation can be found in many publications (e.g. US patent application U.S. Pat. No. 14/774,986) and can be readily adopted for the current application.

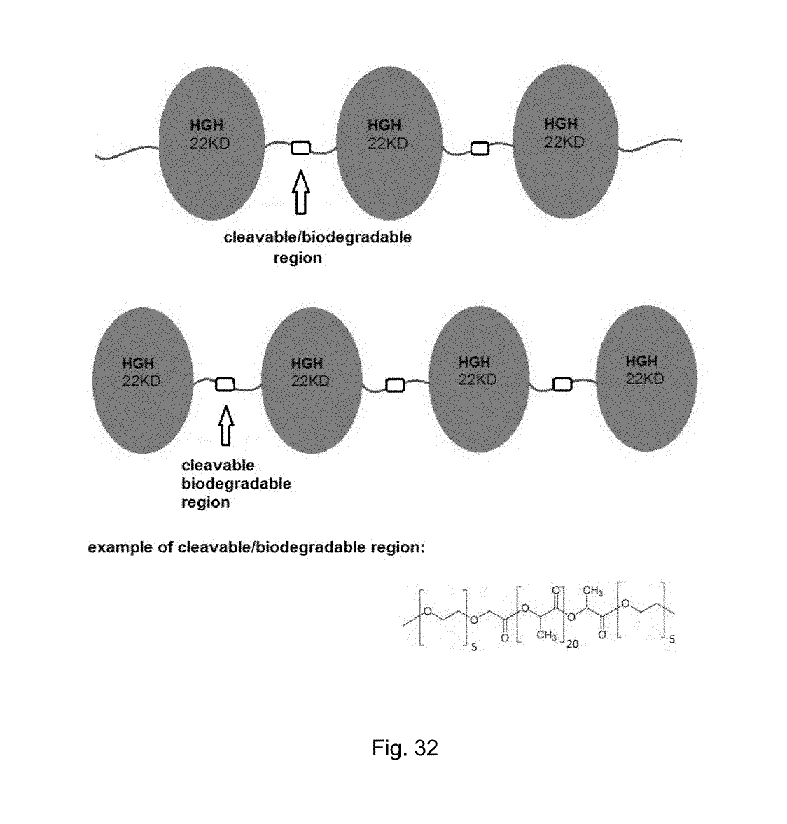

The linker used to construct protein oligomer (e.g. trimer) can also contain one or more cleavable/biodegradable region (FIG. 32), which is essentially a cleavable/biodegradable linker similar to that previously described. This will allow the release of protein monomer or lower degree oligomer slowly in vivo and therefore provide better control on in vivo stability.

This method will reduce renal clearance efficiently with minimal linker (e.g. PEG) content. Small PEG can be used (e.g. 1˜5KD) to achieve total MW of the conjugate >60K to avoid problems associated with high MW PEG, linear structure also increase hydrodynamic size. It can offer better protection against protease degradation. The resulting more drug load and higher activity than mono-pegylated protein due to multivalency will reduce drug amount and volume to improve the comfort of subcutaneous injection. It will provide defined structure and allow site specific conjugation. Higher degree than trimer (e.g. tetramer), biodegradable linker and non-PEG linker (PVA linker, peptide based linker and etc.) can be readily adopted. It is suitable for many proteins with MW 10˜30K. Examples of the protein can be found in well known publications and prior arts, include but not limited EPO, IFN-α, IFN-β, IFN-γ, factor VIII, factor IX, IL-1, IL-2, insulin, insulin analogues, granulocyte colony stimulating factor (GCSF), fibrinogen, thrombopoietin (TPO) and growth hormone releasing hormone (GHRH).

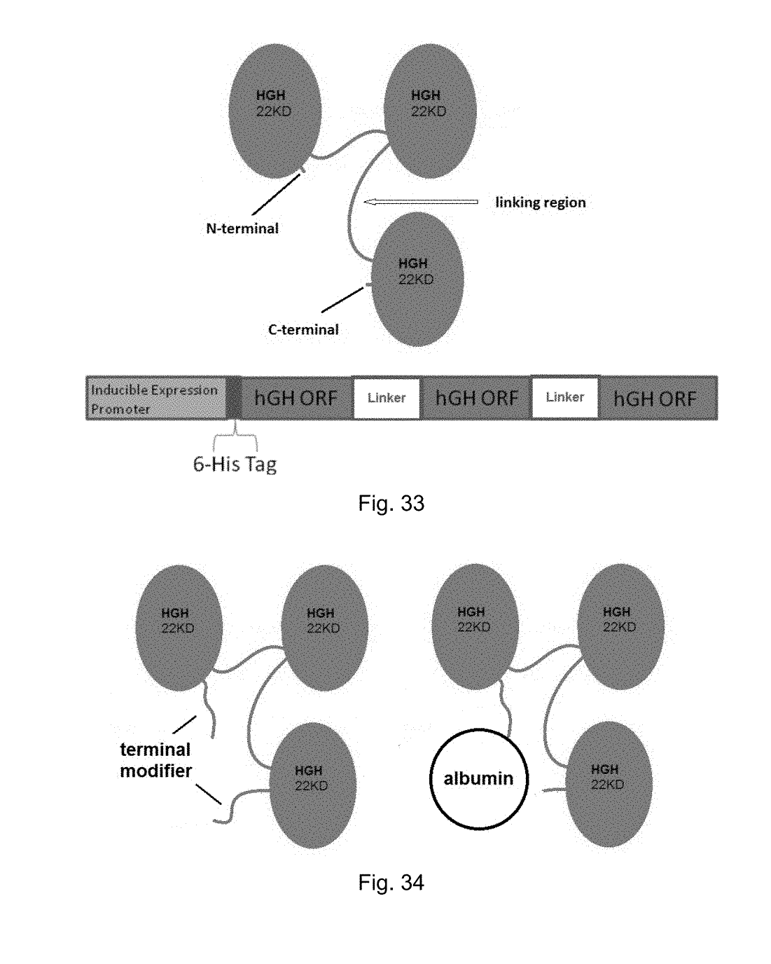

The protein trimer, tetramer or higher degree oligomer can also be produced by expression as recombinant protein, in which each monomer is connected by a flexible peptide linking region from the one's C terminal to another's N terminal. The protein trimer/tetramer or multimer drug is expressed as a whole protein having several monomeric units connected by hydrophilic peptide linking regions, e.g. Asp, Glu, Ser/Gly/Ala rich peptide having 20˜200 AA (amino acids), the negative charged Asp/Glu can inhibit the endocytosis of the protein drug by the cell to reduce receptor mediated clearance, optional protease cleavable sequence can be incorporated into the linking region to adjust its PK. In some embodiments the peptide linker suitable for the current invention contains 10˜150 AA; preferably between 15˜100AA; the sum of glycine (G), alanine (A), serine (S), threonine (T), glutamate (E), aspartate (D), and proline (P) residues constitutes more than about 90% of the total amino acid residues of linker; the sum of glutamate (E) and aspartate (D) residues constitutes more than about 20% of the total amino acid residues of linker. In some embodiments preferably the sum of glutamate (E) and aspartate (D) residues constitutes more than about 30% of the total amino acid residues of linker. Preferably the linker is flexible and displays a random secondary/tertiary structure. Optionally the linker comprises one or more a cleavage sequence (e.g. peptidase/protease cleavage sequence). Preferably the linker constitutes less than about 50% of the total amino acid residues of resulting oligomer. In some embodiments more preferably the linker constitutes less than about 40% of the total amino acid residues of resulting oligomer. In some embodiments more preferably the linker constitutes less than about 30% of the total amino acid residues of resulting oligomer. Preferably the resulting oligomer has a MW>60K. An example of the linker is -GG(ASEGSDEAEGSEASGEGDG)₅-GG (SEQ ID NO: 4). FIG. 33 shows an example of a recombinant HGH trimer and its construction. It can be prepared with E coli expression construct. The Human Growth Hormone Trimer with linker sequence use HGH/Somatropin cDNA identical to HGH from pituitary origin (191 amino acids) Accession Number: DB00052 (BIOD00086, BTD00086). It is tagged with 6-His or other motif for purification. The peptide linker is -GGD(GSEGSEGEASEGSAEGEG)₂-DGG-(SEQ ID NO: 5). The protocol of recombinant protein expression is well known to the skilled in the art and protocols from the publications can be readily adopted for the current invention.

N terminal or C terminal modifier can also be introduced to the oligomer to the N terminal and/or C terminal of the oligomer by recombinant technology. Antibody FC or albumin can also be expressed together with the above oligomer. For example, they can be attached to the N terminal or C terminal of the oligomer by recombinant technology. N terminal and/or C terminal of the oligomer can also be added with modifier sequence such as a flexible peptide sequence similar to the linker using recombinant technology to adjust its in vivo half life (FIG. 34). The alkyl/fatty acid conjugation can also be employed. The protein oligomer generated from recombinant expression can also be further conjugated with half life modifier (e.g. PEG) with site specific conjugation method (e.g. sortase or mTgase conjugation).

The protein oligomer can also be constructed with the combination of recombinant technology and site specific conjugation. First the protein monomer having reactive N terminal and/or C terminal peptide end can be constructed with recombinant technology. Next the reactive N terminal and/or C terminal peptide end can be used as linking region to conjugate with other protein or linkers (e.g. peptide or PEG) with site specific conjugation method. For example the protein monomer can be expressed with reactive end such as Gln/Lys to be used for mTgase based conjugation or LPXTG (SEQ ID NO: 6)/GGGGG (SEQ ID NO: 8) for sortase based conjugation. Optionally a peptide linker can be added between the native protein and the reactive end during the expression. This strategy can avoid the potential folding issue in direct protein oligomer expression. For example, the N terminal of one HGH is added with GGGGG (SEQ ID NO: 8) during expression and the C terminal of another HGH is added with LPETGX (SEQ ID NO: 10) through a flexible peptide linker (e.g. the G/A/D/E rich peptides described above) during expression. Next the two modified HGH monomers are conjugated together with sortase mediated ligation. In another example, a HGH having N terminal GGGGG (SEQ ID NO: 8) and C terminal LPETGX (SEQ ID NO: 10) (e.g. GGGGG (SEQ ID NO: 8)-peptide linker-HGH-peptide linker-LPETGX (SEQ ID NO: 10)) is expressed, next it is used as monomer to prepare oligomer with sortase mediated ligation, the resulting oligomer can be a mixture of HGH oligomer having different degree of polymerization (e.g. dimer, trimer, tetramer and etc.). In another example, excess amount of (e.g. 5˜10 folds) expressed HGH-peptide linker-LPETGX (SEQ ID NO: 10) reacts with expressed GGGGG (SEQ ID NO: 8)-HGH-peptide linker-LPETGX (SEQ ID NO: 10) using sortase mediated ligation to generate HGH-peptide linker-LPET (SEQ ID NO: 11)-GGGGG (SEQ ID NO: 8)-HGH-peptide linker-LPETGX (SEQ ID NO: 10) , which is a HGH dimer. Next the purified HGH dimer is conjugated with GGGG (SEQ ID NO: 12)-HGH using sortase mediated ligation to form the HGH trimer: HGH-peptide linker-LPET (SEQ ID NO: 11)-GGGGG (SEQ ID NO: 8)-HGH-peptide linker-LPET (SEQ ID NO: 11)-GGGGG (SEQ ID NO: 8)-HGH. The expressed HGH can also be conjugated with synthetic molecules (e.g. modified PEG) bearing reactive groups for further conjugation and then the resulting HGH is used to construct oligomer. For example, expressed HGH-(G)n-LPETG (SEQ ID NO: 13) is conjugated with GGGGGG (SEQ ID NO: 14)-PEG-Azide to form the HGH having Azide group with sortase, next the HGH azide is conjugated with a HGH having two alkyne groups (which can be synthesized by coupling alkyne-PEG-NH2 with HGH with mTgase) using click chemistry. The product is a HGH trimer connected with cycloaddtion product of azide with alkyne.

All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well known chemistry, instruments, methods and skills. A skilled person can easily find the knowledge from text books such as the chemistry textbooks, scientific journal papers and other well known reference sources.

1. A method to extend human growth hormone half-life in vivo, comprising:
connecting at least 3 human growth hormone monomers with at least two linkers to form a linear oligomer with one human growth hormone monomer connected with two linkers in the center of the oligomer and two human growth hormone monomers connected to the other ends of the two linkers.

2. The method according to claim 1, wherein the linker is PEG.

3. The method according to claim 1, wherein the linker is peptide.

4. The method according to claim 1, wherein the linker is synthetic polymer.

5. A human growth hormone trimer for extending its half life in vivo, comprising 3 human growth hormone monomers connected with two linkers in a linear form to form a trimer with one human growth hormone monomer in the center of oligomer and two human growth hormones on the two side of the trimer, wherein the human growth hormone monomer in the center are conjugated with two linkers at its glutamine 40 and glutamine 141 residuals respectively.

6. The HGH form according to claim 5, wherein the linker is PEG.

7. The HGH form d according to claim 5, wherein the linker is peptide.

8. An activatable binding aptamer comprising:
a target binding moiety (TBM),
a masking moiety (MM) capable of inhibiting binding of the TBM to a target,
and a cleavable moiety (CM), wherein said CM is positioned in the activatable binding aptamer such that in a cleaved state in the presence of a target, the IBM binds the target, and in an uncleaved state in the presence of the target, binding of the TBM to the target is inhibited by the MM.

9. The aptamer according to claim 9, is further conjugated with toxin.

10. The aptamer according to claim 9, is further conjugated with radioactive material.

11. The aptamer according to claim 9, where in the masking moietyis a nucleotide sequence.

12. The aptamer according to claim 9, where in the CM is an enzyme substrate.


SEQUENCE LISTING (14 sequences)

SEQ ID NO: 1 - protein, 24 aa, Artificial Sequence
  affnity peptide
NIYNCEPANPSEKNSPSTQYCYSI

SEQ ID NO: 2 - protein, 40 aa, Artificial Sequence
  affinity peptide 2
LLGPYELWELSHGGSGGSGGSGGSVPLSLYSGGSGGSGGS

SEQ ID NO: 3 - DNA, 20 bp, Artificial Sequence
  aptamer
cgagaggttggtgtggttgg

SEQ ID NO: 4 - protein, 99 aa, Artificial Sequence
  peptide linker
GGASEGSDEAEGSEASGEGDGASEGSDEAEGSEASGEGDGASEGSDEAEGSEASGEGDGASEGSDEAEGSEASGEGDGASEGSDEAEGSEASGEGDGGG

SEQ ID NO: 5 - protein, 42 aa, Artificial Sequence
  peptide linker 2
GGDGSEGSEGEASEGSAEGEGGSEGSEGEASEGSAEGEGDGG

SEQ ID NO: 6 - protein, 5 aa, Artificial Sequence
  peptide linker 3
  misc_feature (3)..(3): Xaa can be any naturally occurring amino acid
LPXTG

SEQ ID NO: 7 - protein, 6 aa, Artificial Sequence
  sortase substarte 2
  misc_feature (4)..(4): Xaa can be any naturally occurring amino acid
RLPXTG

SEQ ID NO: 8 - protein, 5 aa, Artificial Sequence
  peptide linker 3
GGGGG

SEQ ID NO: 9 - protein, 9 aa, Artificial Sequence
  Peptide linker 4
  misc_feature (3)..(3): Xaa can be any naturally occurring amino acid
LPXTGGGGG

SEQ ID NO: 10 - protein, 6 aa, Artificial Sequence
  Sortase substrate 3
  misc_feature (6)..(6): Xaa can be any naturally occurring amino acid
LPETGX

SEQ ID NO: 11 - protein, 4 aa, Artificial Sequence
  Sortase substrate 4
LPET

SEQ ID NO: 12 - protein, 4 aa, Artificial Sequence
  Peptide linker 5
GGGG

SEQ ID NO: 13 - protein, 5 aa, Artificial Sequence
  Sortase substrate 5
LPETG

SEQ ID NO: 14 - protein, 6 aa, Artificial Sequence
  Peptide linker 6
GGGGGG